(19) United States
(12) Patent Application Publication
(10) Pub. No.: US 2023/0228763 A1
(43) Pub. Date: Jul. 20, 2023

(54) PROCESS FOR DIRECT READOUT OF IMMUNOGLOBULINS

(71) Applicant: Northwestern University, Evanston, IL (US)

(72) Inventor: Neil Lindstrom Kelleher, Evanston, IL (US)

(21) Appl. No.: 18/002,457

(22) PCT Filed: Jun. 18, 2021
(86) PCT No.: PCT/US2021/070729

§ 371 Date: Dec. 19, 2022

Related U.S. Application Data
(60) Provisional application No. 63/194,773, filed on May 28, 2021.
(60) Provisional application No. 63/040,840, filed on Jun. 18, 2020.

(51) Int. Cl. G01N 33/68

(52) U.S. Cl. CPC G01N 33/6854; G01N 33/6848

(57) ABSTRACT

Disclosed herein are methods for the direct readout of proteoforms and complexes thereof, such as immunoglobulins. The method may comprise ionizing a sample with an ionizer, wherein the sample comprises a mixture of different proteoforms or complexes thereof; detecting a multiplicity of ions generated by the ionization of the sample with a current detector; determining ion masses for each of the multiplicity of ions detected with the current detector with a mass analyzer; generating a mass-domain spectrum from the ion masses with the mass analyzer. The method may also comprise determining one or more metrics capturing the heterogeneity or relative abundance of proteoforms.

### CROSS-REFERENCE TO RELATED APPLICATIONS

This application claims the benefit of priority of U.S. Provisional Pat. Application Ser. No. 63/194,773, filed May 28, 2021, and U.S. Provisional Pat. Application Ser. No. 63/040,840, filed Jun. 18, 2020, the contents of each are incorporated by reference in their entirety.

### STATEMENT REGARDING FEDERALLY SPONSORED RESEARCH

This invention was made with government support under GM108569 awarded by National Institutes of Health. The government has certain rights in the invention.

### REFERENCE TO A SEQUENCE LISTING

This application is being filed electronically via EFS-Web and includes an electronically submitted Sequence Listing in .txt format. The .txt file contains a sequence listing entitled “702581_01972_ST25.txt” which is 3.58 KB in size and was created on Jun. 18, 2021. The Sequence Listing contained in this .txt file is part of the specification and is hereby incorporated by reference herein in its entirety.

### BACKGROUND OF THE INVENTION

Antibody testing in blood has drastic differences in sensitivity and specificity depending on the manufacturer. High false positive rates are likely due to cross-reactivity of antibodies targeting other coronaviruses, such as those generated against the common cold. In order for these tests to become more reliable and allow for improved public health decisions, the antibody response must be more thoroughly investigated with more specific technologies, thereby enabling improved accuracy of serological testing and epidemiology (“serosurveillance”).

Since late 2019, severe acute respiratory syndrome coronavirus 2 (SARS-CoV-2) and its associated disease COVID-19¹ have been at the center of a global pandemic affecting more than 150 million people, especially immunocompromised individuals, the elderly, and individuals with pre-existing conditions². This has resulted in >3 million deaths worldwide, including ~600,000 in the USA where COVID-19 became a leading cause of death^{3,4}. Unfortunately, the global death toll is still rising due to delays in vaccination and the emergence of more transmissible variants of SARS-CoV-2, against which the available vaccines and antibody-based therapeutics may be less effective⁴.

Most COVID-19 patients develop antibodies against SARS-CoV-2 within a few weeks after infection^{5,6}, commonly recognizing targets such as viral envelope, nucleocapsid, and spike proteins. In particular, the spike protein receptor-binding domain (RBD) is a robust immunogenic target that has been the focus of many antibody-based diagnostics and therapeutics against SARS-CoV-2⁷.

Generally, standard serology tests (i.e., ELISA and lateral flow assay) can reliably detect whether a patient possesses antibodies against a target, but they do not capture the clonality and makeup of an entire immunoglobulin (Ig) repertoire. As a result, there is a need for more precise assessment of immune status at single clone resolution to significantly improve serological testing capabilities.

### BRIEF SUMMARY OF THE INVENTION

Disclosed herein are methods for the direct readout of proteoforms and complexes thereof, such as immunoglobulins. One aspect of the invention provides for a method for immunoglobulin repertoire profiling. The method may comprise reducing a mixture of immunoglobulins in a sample, the sample optionally further comprising a standard immunoglobulin, thereby obtaining a second sample comprising a mixture of light chains and heavy chains or light chains and Fd domains; ionizing the second sample with an ionizer; detecting a multiplicity of ions generated by the ionization of the second sample with a current detector; determining ion masses for each of the multiplicity of ions detected with the current detector with a mass analyzer; generating a mass-domain spectrum from the ion masses with the mass analyzer; and determining one or more metrics capturing the heterogeneity or relative abundance of individual immunoglobulins. In some embodiments, an Ion Titer (IT), a degree of clonality (DoC), a spectral correlation coefficient, or any combination thereof is determined.

Another aspect of the invention provides for a method for identifying a target proteoform or complex there. The method may comprise ionizing a sample with an ionizer, wherein the sample comprises a mixture of different proteoforms or complexes thereof; detecting a multiplicity of ions generated by the ionization of the sample with a current detector; determining ion masses for each of the multiplicity of ions detected with the current detector with a mass analyzer; generating a mass-domain spectrum from the ion masses with the mass analyzer; obtaining a spectral signature for a target ion mass of the target proteoform or complex thereof with a gene analyzer; and identifying the presence or absence of the spectral signature for the target ion mass in the mass-domain spectrum.

These and other aspects of the invention will be further described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 provides a schematic illustration of the platform for identifying proteoforms, including immunoglobulins such as IgG.

FIG. 2 illustrates the I²MS process.

FIG. 3. Overview of sample preparation. Blood is acquired of COVID-19 positive patients. From plasma, antibodies are enriched for SARS-CoV-2 spike protein antibodies. Samples are cleaned up on SampleStream before I²MS. For MS1 pattern matching and fragment profiling, antibodies are digested and reduced on SampleStream prior to analysis.

FIG. 4. I²MS profile of IVIG. IVIG was measured directly by I²MS from healthy patients. Intact immunoglobulins are shown in the predicted range. IgG’s retain their glycosylation and are pooled from >2 dozen donors; a readout in <1 hour from sample to data was readily achieved.

FIG. 5. A histogram showing the density of predicted mass values (1 Da bins) for the IgG V_{H} region of 5,643 human plasma cells as determined by scRNASeq on a blood sample from a single donor.

FIG. 6. Amino acid motif of neutralizing antibodies. Convergent CDR3H sequence of SARS-CoV-2 nAb’s based on primary literature or B cell sequencing.

FIGS. 7A-7C. Ig-MS, a new platform for COVID-19 serology. FIG. 7A, Overview of sample preparation and readout. Antibodies against the RBD domain of the SARS-CoV-2 spike protein are enriched from plasma of COVID-19 patients and controls using magnetic beads. Before elution, Spike RBD-specific Igs are either eluted (workflow 1) or treated with the IdeS enzyme to remove the Fc domain of the HC (workflow 2). The eluates are then reduced to obtain LC and HC (workflow 1) or LC and Fd (workflow 2), which are analyzed by individual ion mass spectrometry (I²MS). FIG. 7A, Western blot showing the enrichment of Ig targeting SARS-CoV-2 Spike RBD from P1877 using workflow 1. FIG. 7C, Example data set and metrics obtained from Ig-MS of the plasma from a COVID-19 convalescent patient (COVID-19_3) (only LC is shown).

FIGS. 8A-8B. Expression validation of RBD region from the SARS-CoV-2 Spike protein. FIG. 8A, A peptide map of Spike-RBD from bottom-up proteomics showing 100% sequence coverage. FIG. 8B, Western-blot using the mAb CR3022 standard monoclonal antibody that recognizes RBD as primary antibody.

FIGS. 9A-9B. Western blot showing isotypes and IgG subclasses of the antibodies enriched with Ig-MS assay. RBD-specific antibodies were immunoprecipitated from the plasma of a COVID-19 convalescent patient with magnetic beads conjugated with the recombinant RBD protein and separated via SDS-PAGE. Proteins were transferred to nitrocellulose and probed with antibodies recognizing human (FIG. 9A) antibody isotypes IgA, IgD, IgE, and IgM or (FIG. 9B) IgG subclasses IgG1, IgG2, IgG3, and IgG4. Isotype-specific antibodies were used as positive controls, and pooled human isotype antibodies excluding the isotype of interest were used as negative controls.

FIG. 10. Proteoform annotation of the CR3022 standard monoclonal antibody. Mass spectrum acquired by Ig-MS in black and the theoretical mass distribution in red for the light chain and glycoproteoforms on the heavy chain of the antibody, produced in Nicotiana bethamiana (tobacco plant).

FIGS. 11A-11B. Titration curve for Ig-MS to estimate the limit of detection (LOD) for NIST standard monoclonal antibody. Standard curves for (FIG. 11A) light chain (LC) and (FIG. 11B) heavy chain (HC) with observed intensities and concentrations are shown. The LOD for the HC was ~100 nM, whereas that for the LC was not reached at the 10 nM data point; the LC LOD is estimated at <1 nM.

FIGS. 12A-12B. Ig-MS readout of light chain region for P1877. FIG. 12A, Mass spectrum of all light chains isolated from plasma sample P1877. FIG. 12B, Zoom in at 10% intensity to show LC peaks that correspond to 1% of the standard intensity. The peak highlighted in blue is the spiked-in mAb CR3022 at 100 ng.

FIG. 13. Ig-MS readouts from Workflow 1 (Ig Light and Heavy Chains). Results from plasma of ten COVID-19 convalescent patients, including seven hospitalized patients (red) and three outpatients (purple). Also, Ig-MS was applied to the plasma of three uninfected individuals, and a pool of plasma collected before the emergence of SARS-CoV-2 (green). Monoclonal antibody CR3022 anti-SARS-CoV-2-RBD (blue) was used as positive control and is highlighted in gray.

FIG. 14. Ig-MS readouts of Light Chains obtained using Workflow 1. Plasma of ten COVID-19 convalescent patients, including seven hospitalized patients (red) and three outpatients (purple). In parallel, the assay was applied to the plasma of three uninfected individuals, and a pool of plasma collected before the emergence of SARS-CoV-2 (green). Monoclonal antibody CR3022 anti-SARS-CoV-2-RBD (blue), used as a positive control for quantitation, is highlighted in gray.

FIG. 15. Ig-MS readouts of Heavy Chains obtained using Workflow 1. The plasma of ten COVID-19 convalescent patients, including seven hospitalized patients (red) and three outpatients (purple). Also, Ig-MS was applied to the plasma of three uninfected individuals, and a pool of plasma collected before the emergence of SARS-CoV-2 (green). Monoclonal antibody CR3022 anti-SARS-CoV-2-RBD (blue) spectrum was used as a positive control.

FIGS. 16A-16L. Ig-MS readouts from workflow 1 and 2 on a COVID-19 cohort. Results for the LC spectral region from workflow 1 (FIGS. 16A-16D) and for the LC and Fd fragments from workflow 2 (FIGS. 16E-16H); ion titers (IT), and degree of clonality (DoC) are shown for two hospitalized patients (FIGS. 16A, 16E and FIGS. 16B, 16F, red) and one outpatient (FIGS. 16C, 16G, purple). The standard mAb (CR3022) that binds SARS-CoV-2-RBD (FIGS. 16D, 16H, blue) was used as positive control (highlighted with gray vertical bar). FIG. 16I, Comparison of Ig-MS patterns between the LC of a single patient obtained with the two workflows. FIG. 16J, IT and DoC values obtained by both workflows of Ig-MS for three outpatients and seven hospitalized patients. Analyses were done in triplicate. Correlation of IT (FIG. 16K) and DoC (FIG. 16L) values from Ig-MS workflows with RBD-binding by ELISA, bioluminescent in vitro diagnostic (Promega), and surrogate virus neutralization. Shown are the Pearson correlation coefficient (R) and P-value (P).

FIG. 17. Ig-MS results from Workflow 2 (light chain and Fd fragment from the heavy chain). The plasma of ten COVID-19 convalescent patients, including seven hospitalized patients (red) and three outpatients (purple). In parallel, the assay was applied to the plasma of three uninfected individuals and a pool of plasma collected before the emergence of SARS-CoV-2 (green). Monoclonal antibody CR3022 anti-SARS-CoV-2-RBD (blue) was used as a positive control.

FIGS. 18A-18B. Comparison of ion titers between Workflow 1 and Workflow 2. FIG. 18A, Box-and-whisker plot demonstrating the spread of ion titers obtained with the two Ig-MS workflows for light chain repertoires from ten COVID-19 patients. FIG. 18B, Correlation between ion titers obtained with the two workflows.

FIGS. 19A-19C. Anti-RBD antibody and neutralization titers. Titers and neutralization capacity of anti-RBD antibodies from COVID-19 outpatients (purple) and hospitalized patients (red) calculated by ELISA (FIG. 19A), bioluminescent in vitro diagnostic (FIG. 19B), and surrogate virus neutralization (FIG. 19C).

FIG. 20. Correlation of Ion titer (IT) and Degree of Clonality (DoC) metrics from Ig-MS with days after infection. Shown are the Pearson correlation coefficient (R) of the Ion Titer and the Degree of Clonality from Workflows 1 and 2 against days after the infection. P-value (p).

FIG. 21. Comparison of bulk IgG glycosylation profiles. COVID-19 patients hospitalized (red) and outpatients(purple) with those of uninfected individuals (green). Statistical significance was calculated by one-way ANOVA with Tukey’s multiple comparison test (*, p<0.05; **, p<0.01).

FIGS. 22A-22B. Comparison of total glycan composition. Percentage of total (FIG. 22A) fucosylated and (FIG. 22B) afucosylated glycans observed on COVID-19 patients hospitalized (red) and outpatients(purple) with those of uninfected individuals (green). Statistical significance was calculated by one-way ANOVA with Tukey’s multiple comparison test (*, p<0.05).

FIG. 23. Ig-MS readouts from workflow 1 on the vaccinated cohort. Results for the LC spectral region from workflow 1 for (a) uninfected before the vaccination (time 0), (b) vaccinated at 20 days after the first shot (time 1), and (c) vaccinated at 28 days after the second shot (time 2). (d) IT and (e) DoC values obtained by both workflows of Ig-MS for time-points 1 and 2. (f) Bioluminescent in vitro diagnostic (Promega) titer of anti-RBD antibodies

FIG. 24 shows Ig-MS spectra from Myasthenia Gravis (MG) patients versus a healthy donor with MuSK as the proposed antigen. HD57: healthy donor. MYG28: AChR MG. MYG69: MuSK MG.

FIG. 25 shows Ig-MS spectra from MYG patents and a healthy donor at various time points showing the MuSK patients retains the extra peak, indicated by an arrow, over several months.

### DETAILED DESCRIPTION OF THE INVENTION

Disclosed herein are methods for the direct readout of proteoforms and complexes thereof, such as immunoglobulins. The disclosed methods allow for the identification of full-length IgG-type antibodies, or their heavy chains, light chains, of Fd domains, without the need to fragment the proteoforms prior to detection or complex analysis to reconstruct the full length proteoform from the fragments. As a result, the disclosed technology allows for profiling the immunoglobulin repertoire and the identification of specific antibodies generated by the human body in response to an infection, such as a SARS-CoV-2 infection, or vaccination with high resolution and molecular precision. The technology also allows for identification of aberrant IgGs and other immunoglobulin subtypes that are associated with autoimmune disorders. The disclosed methods also allow for the identification of donors capable of providing therapeutic immunoglobulins for specific immunoglobulin or convalescent plasma therapies, enabling the production of such proteoform products. The technology also allows for screening for drugs capable of inducing immunoglobulins to bind tighter to antigens or carry therapeutic agent or other cargo along with the immunoglobulin for anti-viral activity.

Moreover, profiling the IgG repertoire (e.g., from recovered COVID-19 patients), and those both asymptomatic and symptomatic can serve as a counterpart to serosurveillance via ELISA-based test kits. This could provide vital information by providing direct monitoring of IgG responses at single clone resolution to help assess false negative and false positives. The same approach can be used for IvIG samples, samples from plasma donors, and the identification (or characterization) of IgG-based therapies.

One aspect of the invention is a method for identifying a target proteoform or a complex thereof, such as an immunoglobulin or its heavy chain, light chain, or Fd domain. As used herein, “proteoform” refers to all of the different molecular forms in which the protein product of a single gene can be found, including changes due to genetic variations, alternatively spliced RNA transcripts and posttranslational modifications, and the like. Suitably, the proteoform is an immunoglobulin, such as an IgG. IgA, IgE, or IgM.

Although the methods may suitably be used to identify monocolonal immunoglobulins, an advantage of the present technology is that it can be used to identify immunoglobulins with far polyclonality because the individual, or single-ion approach to disclosed herein functions on highly polydisperse proteins. In contrast, “ensemble” methods, such as classical mass spectroscopy approaches, provide no data whatsoever on mixtures of such complexity.

Antibodies are produced by a developmentally ordered series of somatic gene rearrangement events that occur exclusively in developing B cells and continue throughout the life of an organism. Antibodies consist of heavy (µ, α, γ, δ, ε) and light chains (κ, λ), which are linked by disulfide bonds. The intact antibody contains variable and constant domains. Antigen binding occurs in the variable domain, which is generated by recombination of a finite set of tandemly arranged variable (V), diversity (D) and joining (J) germline gene segments. This process, called VDJ recombination, often results in the addition and deletion of nucleotides at the junctions between ligated gene segments. More specifically, DNA exonucleases can trim the ends of the gene segments, and DNA polymerases and transferases can randomly insert templated palindromic or nontemplated nucleotides, respectively.

During B-cell development, immunoglobulin heavy (IgH) chain gene recombination typically occurs before immunoglobulin light (IgL) chain gene recombination. If both IgH and IgL genes are productively rearranged, the fully assembled antibody heterodimer is expressed on the surface of the B cell. In B cells bearing productively rearranged antibodies, the process of allelic exclusion (and locus exclusion in the case of IgL) ensures that each B cell expresses a single antibody⁵. After passage through several developmental checkpoints, newly generated mature IgM⁺IgD⁺ B cells form the naive B cell (and, therefore, naive antibody) repertoire. Most of the diversity in the naive antibody repertoire is concentrated at the site of IgH VDJ gene segment ligation, also known as the IgH complementarity-determining region 3 (CDR-H3). Because of the combinatorial and nontemplated nature of the mechanisms that generate the CDR-H3, it is the most diverse component in terms of length and sequence of the antibody H-chain repertoire and is a principal determinant of antibody specificity. Nonetheless, there are instances where antigen specificity is dictated solely or predominantly by the L chain.

When a B cell encounters antigen in an environment that provides requisite co-stimulatory signals and T-cell help, BCR stimulation induces B-cell proliferation. This process, known as B-cell clonal expansion, occurs primarily in highly organized areas of secondary lymphoid organs (e.g., spleen, lymph nodes and Peyer’s patches⁹) referred to as germinal centers. Clonal expansion is followed by somatic hypermutation of the variable domains of antibodies; this is mediated by activation-induced cytidine deaminase. B cells expressing BCRs bearing somatic mutations that increase affinity for antigen outcompete other B cells for access to antigen. As a result, the B cells bearing the highest-affinity antibodies undergo preferential expansion and survival, a process referred to as affinity maturation. Somatic hypermutation also results in sequence diversification of the CDR-H1 and CDR-H2 hypervariable regions and of the framework 3 (FR3) region. Activation-induced cytidine deaminase also mediates class-switch recombination, which generates antibodies bearing different constant regions. B cells expressing somatically mutated, high-antigen-affinity BCRs can differentiate into long-lived memory B cells, capable of mediating rapid recall responses to the same antigen, or into terminally differentiated plasma cells; the latter downregulate BCR expression, establish residency in the bone marrow, gut lamina propia (and, to a smaller degree, in secondary lymphoid tissues), and secrete protective antibodies at extremely high rates estimated at 10,000-20,000 antibody molecules per second. Antibody production by long-lived plasma cells in the bone marrow is postulated to proceed for very long times, possibly throughout the entire lifetime of the organism.

Diversity in the primary antibody repertoire before exogenous antigen exposure stems from the allelic diversity in immunoglobulin gene segments, combinatorial diversity introduced during somatic recombination, junctional diversity caused by the imprecision of the recombination process, pairing of IgH and IgL polypeptide chains, and receptor editing, wherein the existing V-gene segment is replaced with another. In addition, VH replacement, a process resulting from the presence of a cryptic recombination signal sequence in FR3, might influence as much as 5-12% of the human primary B-cell antibody repertoire. Diversification of the post-antigen-stimulation secondary antibody repertoire stems from somatic hypermutation and class-switch recombination.

In contrast to a “bottom-up” proteomics approach that require the digestion or fragmentation of proteoforms into peptide fragements, the present technology is a “top-down” approach that avoids the digestion of proteoforms and the preoteoform is directly identified from an intact protein. The advantage of the present approach is that the top-down approach provides the richest data for both precise identification (that is, the specific gene in a higher eukaryote that encodes the protein measured) and full characterization of molecular composition. However, it is considerably more challenging to execute than the bottom-up approach because of the complexity of the data generated and various technical limitations.

The method comprises the use of an ionizer, current detector, mass analyzer, and gene analyzer to identify the present or absence of a target proteoform or a complex thereof. As used herein, “target proteoform” refers to the proteofrom sought to be identified by any of the methods disclosed herein. In some embodiments, the target proteoform is a proteoform indicative of an immunological response to an antigen, such as an immunoglobulin or, more particularly, IgG. In some embodiments, the target proteoform is a therapeutically effective proteoform, such as those that may be useful for the preparation of proteome products capable of treating a subject in need of the proteome product. In some embodiments, a proteome product comprises an immunoglobulin capable of use for an immunoglobulin therapy.

The method for identifying the target proteoform or a complex thereof comprises ionizing a sample with an ionizer and detecting a multiplicity of ions generated by the ionization of the sample with a current detector. Suitably the sample may comprise a mixture of different full-length proteoforms or complexes thereof, such as bodily fluid samples that may be obtained from a subject or a donor candidate. As used herein, “donor candidate” is a subject that may possess a preteoform useful for the preparation of a proteome product. In some embodiments, the ionizer and the current detector comprise components of a mass spectrometer.

From the detected ions, the mass of each of the ions may be determined with a mass analyzer. As used herein, “mass analyzer” may include a programmable processor or combination of processors, such as central processing units (CPUs), graphics processing units (GPUs), Field Programmable Gate Arrays (FPGAs), Application-Specific Integrated Circuits (ASICs) and the like. As such, the mass analyzer may be configured to execute instructions stored in a non-transitory computer readable-media. In this regard, the mass analyzer may be a computer, workstation, laptop or other general-purpose computing device. Additionally or alternatively, the controller may also include one or more dedicated processing units or modules that may be configured (e.g. hardwired, or pre-programmed) to carry out steps, in accordance with aspects of the present disclosure.

In an embodiment of the invention, the mass analyzer is configured to receive a signal from the current detector to determine the ion masses and generate a mass-domain spectrum. Mass spectrometry has used ions to measure the mass-to-charge (m/z) ratio of molecules once lifted into the gas phase. Denatured and native electrospray ionization of intact proteins and their complexes pose many complications due to sample heterogeneity and large charge-state envelopes in the m/z domain. To simplify analysis, charge detection mass spectrometry (CDMS) has enabled the generation of true mass spectra with the direct readout of an ion’s integer charge value. An exemplary approach for determining the ion masses and generating a mass-domain spectrum includes an individual ion mass spectrometry (I²MS) approach. I²MS allows for measuring complex proteoform mixtures and their complexes without the need to separate the proteoforms prior to identification. [Kafader, J.O. Nat Methods 17, 391-394 (2020)]

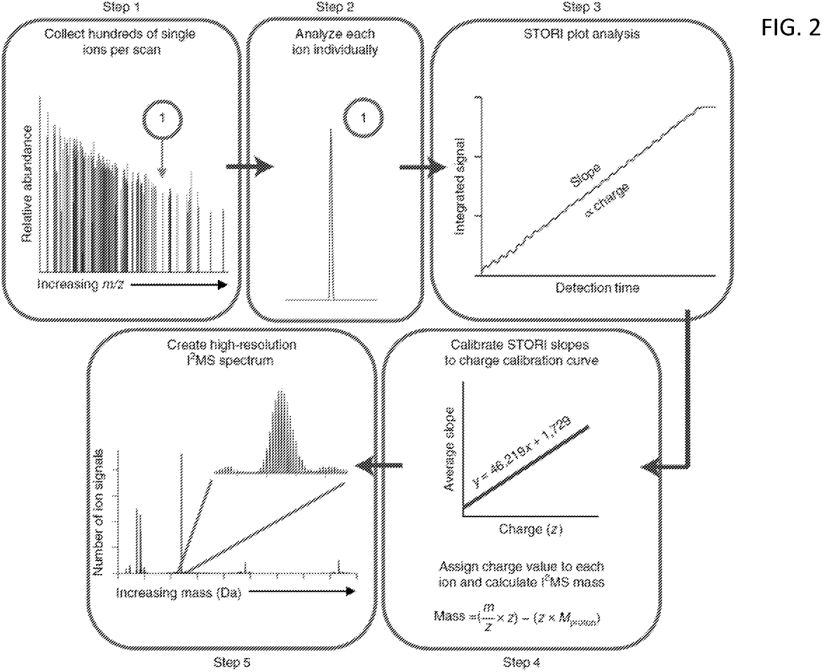

In step 1 (FIG. 2) of the I²MS approach, ~120 ions may be observed per acquisition in a random-style trapping event. Parallelizing ion observation to >100 ions per acquisition decreases the data acquisition burden by ~100-fold over current CDMS techniques. Owing to the low ion count needed for I²MS, protein solutions can be diluted down by about three orders of magnitude (into the high pM to low nM range). In step 2 (FIG. 2), the frequency of each ion signal is determined and analyzed independently. At this processing stage, precise information for each ion, including frequency, intensity and m/z value, is established. Step 3 (FIG. 2) determines the ion’s signal strength using a data-plotting and data-analysis process that assesses the current induced by an ion on the detection electrodes as a function of acquisition time. For simplicity, we call this signal strength determination the selective temporal overview of resonant ions (STORI) process, with the slope of a STORI plot being proportional to the charge of the ion (step 3, FIG. 2). In step 4, the charge of the ion is determined by a slope-to-charge calibration function. The STORI slope of an ion with an unknown charge is assigned the closest integer charge state on the calibration function. Each calibration function was created just once for each of the two Q-Exactivestyle instruments used in this study. Using the integer charge (z) and m/z, it is possible to determine the mass of each ion and produce a spectrum in the true mass domain with different spectral properties and increased resolution via centroiding and binning individual ion signals.

This approach allows for the direct readout of a mixture of heavy chains, light chains, or Fd domains resulting from disulfide reduction of an IgG. The remarkably clean baselines of I²MS spectra proved highly insensitive to partially desolvated ions, which extended the dynamic range for intact mass determination even when complexity was high. In essence, I2MS transforms the instrument into an actual mass spectrometer, removing the normal requirement for an inference step.

In some embodiments, the method may also include obtaining a spectral signature for a target ion mass of the target proteoform or the complex thereof with a gene analyzer. As used herein, “gene analyzer” may include a programmable processor or combination of processors, such as central processing units (CPUs), graphics processing units (GPUs), Field Programmable Gate Arrays (FPGAs), Application-Specific Integrated Circuits (ASICs) and the like. As such, the gene analyzer may be configured to execute instructions stored in a non-transitory computer readable-media. In this regard, the controller may be a computer, workstation, laptop or other general-purpose computing device. Additionally or alternatively, the gene analyzer may also include one or more dedicated processing units or modules that may be configured (e.g. hardwired, or pre-programmed) to carry out steps, in accordance with aspects of the present disclosure.

In an embodiment of the invention, the gene analyzer is configured to determine a mass for the target proteoform or the complex there from a nucleic acid sequence corresponding to the target proteoform or complex thereof. The mass determined by the gene analyze may be used to obtain a spectral signature for the target ion mass, which allows for the identification of the presence or absences of the target ion mass in the mass-domain spectrum generated by the mass analyzer. In some embodiments, the nucleic acid sequences corresponding to the target proteoform may be obtained from the sample being detected. For example, a sample obtained from a subject or donor candidate may be fractionated, and one fraction used for the determination of mass-domain spectrum and another fraction used to sequence the nucleic acids. In other embodiments, the nucleic acid sequences corresponding to the target proteoform may be obtained from a database containing nucleic acid sequence information.

The present methods may be used in combination with preteoform sequencing, such as immunoglobulin sequencing (Ig-seq). Ig-seq allows for proteomic identification of antibodies from biological fluids, such as blood, serum, plasma, or cerebral spinal fluid. [Georgiou, G. Nat Biotechnol. 2014; 32(2): 158-168] Suitably, the antibodies may also be collected from tissue samples, such as lung tissue samples. The advent of antibody discovery by proteomic mining of the serum antibody response has in turn opened the way for the isolation of biologically relevant antibodies from convalescent patients. Finding the serum antibodies that were responsible for the resolution of disease states in patients is of great relevance for drug discovery because such antibodies will have already been established to be of therapeutic value. Information gained from Ig-seq can be applied to detect B-cell malignancies with high sensitivity, to discover antibodies specific for antigens of interest, to guide vaccine development and to understand autoimmunity.

In some embodiments, the method further comprises isolating one or more cells from the sample comprising the target proteoform or a complex thereof and sequencing a nucleic acid from the isolated cells, wherein the sequenced nucleic acid corresponds to the target proteoform or a complex thereof. Suitably the cells are peripheral blood mononuclear cells (PBMCs), such as B cells.

Underlying sequence information is essential for properly distinguishing isobaric antibodies. Ig-Seq allows the generation of V_{H}/V_{L} chains repertoires typically of the order of 10⁶-10⁷ nucleotide sequence. More complex single cell sequencing methods allows for the preservation of the V_{H}-V_{L} pairings, but yielding smaller datasets. In order to determine the underlying sequence for the antibodies detected by I²MS in Aim 2A, fluorescence-activated cell sorting (FACS) may be used to isolate memory B-cells against SARS-CoV-2 spike protein using a method similar to recent reports, with memory B cells isolated using typical markers (CD19+CD27+IgD-IgM-). The final step for isolating memory B cells specific for SARS-CoV-2 employs a fluorescently tagged spike protein RBD, allowing for isolation of cells expressing antibodies which would be detected by enrichment and mass spectrometry. Single-cell RNA-Seq of PCR-amplified immunoglobulin regions (Ig-Seq) may be used to determine the sequences of heavy and light chains to provide a list of candidate Ig heavy and light chain amino acid sequences.

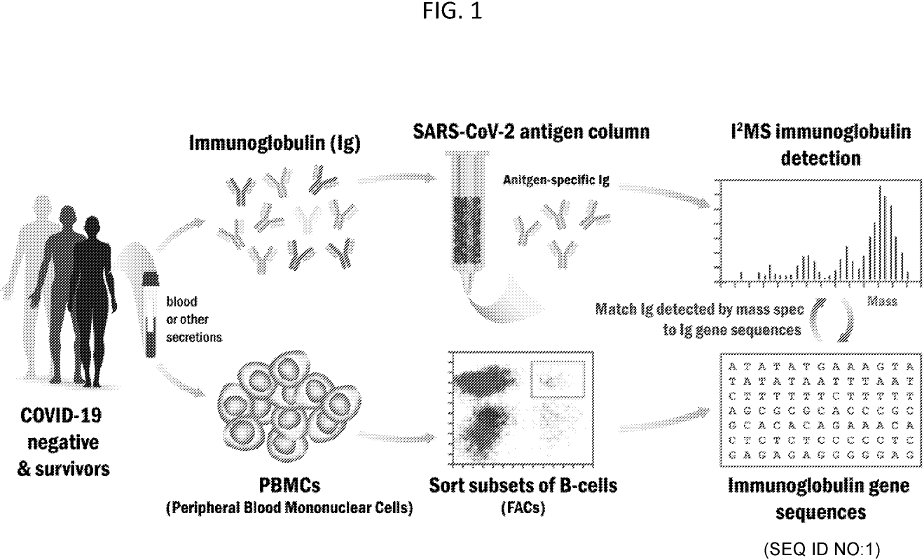

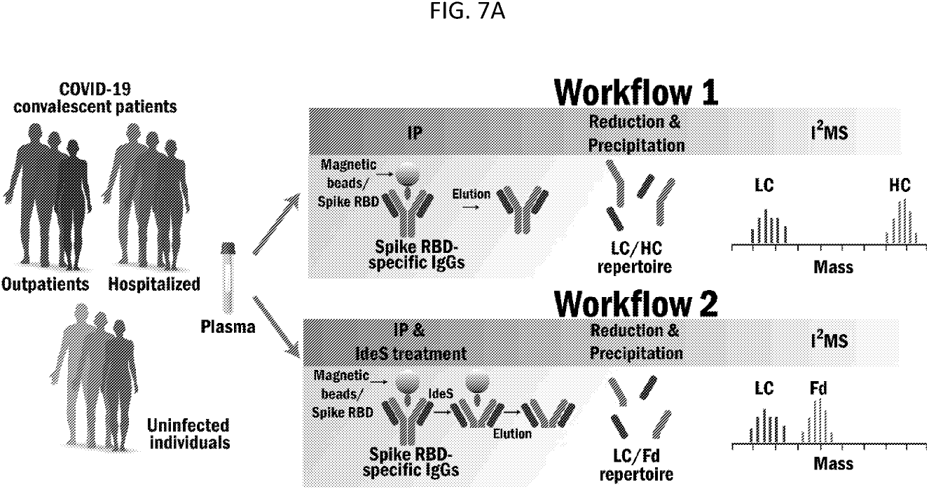

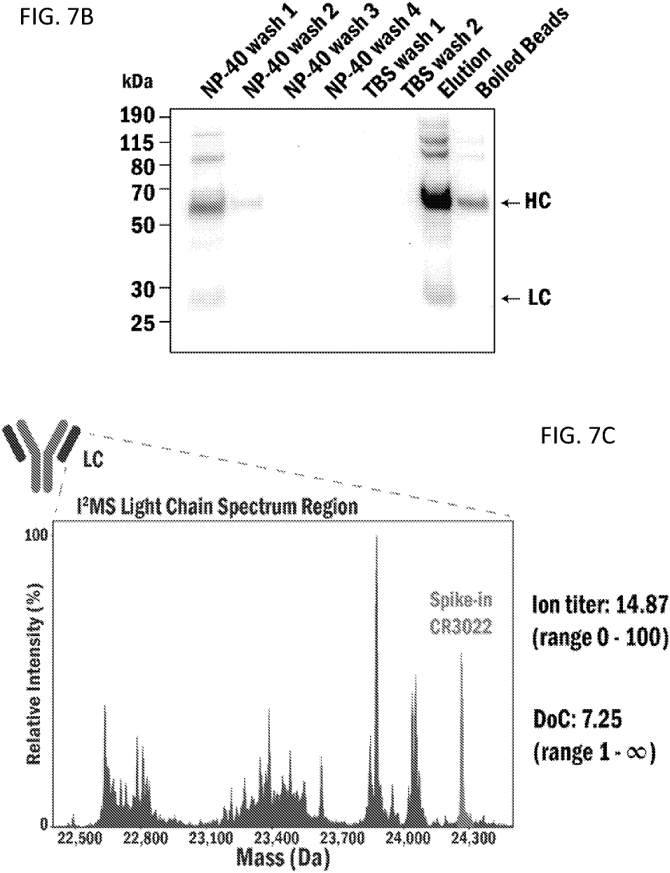

In some embodiments, the sample may be fractionated by one or more means. As used herein, “fractionated” means to separate one or more components of the sample. In some embodiments, the sample may be fractioned by a mechanical fractionation technique, such a centrifugation or membrane size exclusion. In other embodiments, the sample may be fractioned by a chemical fractionation technique, such as precipitation (such as immunoprecipitation), binding affinity, chromatography, or washing, such as by 20-100x concentration or buffer exchange techniques. In some embodiments, the sample may be fractionated to separate proteoforms or complexes thereof from cellular matter, such as illustrated in FIGS. 1 or 7A. In other embodiments, the sample may be fractionated to separate some proteoforms from other proteoforms, such as by antigen binding as illustrated in FIGS. 1 or 7A.

In some embodiments, proteoforms are chemically modified prior to ionization. Suitably the proteoforms may be deglycosylated, e.g., PNGaseF, disulfide bonds may be reduced, or digested with a protease, e.g., papain, IdeS, Gengkis Khan. As a result, the method may directly detect a proteoform complex, such as an IgG. In another embodiment, the method directly measures the individual proteoform components of a complex, such as the heavy and light chains of an IgG.

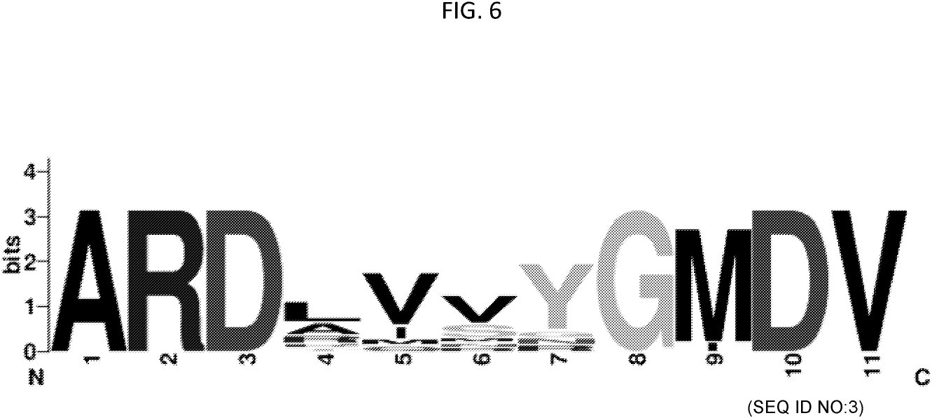

To produce antibody fragments which are amenable to fragmentation of the CDR3 region, a papain digestion and disulfide reduction step can be performed. These steps will produce free Fab fragments containing the CDR3 regions. By doing tandem mass spectrometry and looking at specific subsequences, like the sequence motif from the high value CDR3 region (See, FIG. 6), and because the method uses big, i.e., 25-50 kDa intact ions, we will get all the CDR2 and CDR1 regions near the N-terminus of the IgG antibodies as well. In addition, the method may employ multiple fragmentation types, by which plasma can be monitored for the presence of antibodies containing the amino acid motif of nAb’s. For candidate nAb identification from protein fragmentation data, N-terminal fragment ions should elucidate antibody specificity. According to IMGT nomenclature the CDR3 is between a conserved Cys104 and a conserved Trp118 or Phe118. Using the sequences for SARS-CoV-2 nAb’s collected in the Oxford CoV-AbDab database, we determined that the CDR3 will likely be between residues 95-128 from the N-terminus. This is well within the limits of measurability by top-down proteomics at ~11-13 kDa, strongly indicating that specific antibodies can be identified from IgG fragmentation data.

In some embodiments, the method may also include determining one or metrics capturing the heterogeneity or relative abundance of individual immunoglobulins. The methods disclosed herein allow for new serology methods that provides a readout of all immunoglobulins produced against a specific antigen with molecular resolution using proteomics methods. Such methods are capable of capturing the complete pattern of the light chain and heavy chains of immunoglobulins produced by the adaptive immune system. Moreover, the methods allow for determining quantitative patterns containing all variability from the 3 CDRs regions from light and heavy chains in a single analysis.

The presently disclosed methods fractionates immunoglobulins against a specific target from the plasma of patients and controllably breaks them down into their heavy and light chains (HC and LC). Once created, HC and LC fragments are analyzed by individual ion mass spectrometry (I²MS). The presently disclosed methods provide compositional profiles for immunoglobulin repertoires, their degree of clonality, and titers. As a result, the disclosed methods provide high-value clinical correlates of viral neutralization, the presence, extent, and course of the disease, and/or assess the degree of protection after vaccination.

In some embodiments, samples comprising a mixture of immunoglobulins may be fractioned, such as through immunoprecipitation, to isolate immunoglobulins having an affinity for an antigen. The fractionated sample may be combined with a standard immunoglobulin and reduced or denatured in the presence of reducing or denaturing agent to liberate heavy chain and light chain proteoforms. The method may optionally further comprise chemically modifying the proteoforms, including digesting the proteoforms with a protease such as IdeS. When the method comprises a chemical modification, reducing or denaturing the chemically modified proteoforms allows for the liberation so light chain and Fd domains. Either of these workflows captures the compound of all the composition of all three Complementarity-Determining Regions (CDRs)

In some embodiments, an Ion Titer (IT) is determined. The Ion Titer (IT) is similar to an ELISA titer and uses the intensity of the LC of a standard immunoglobulin as a reference, and combines intensity of all other LC peaks and ranges from 0 to 100. The IT may be determined from the peaks of the mass-domain spectrum through the use of Equation 1.

In some embodiments, a degree of clonality (DoC) is determined. the Degree of Clonality (DoC), a measure of the complexity of all LC clones (proteoforms) in the mixture and ranges from 1 to infinity, with higher numbers reflecting the presence of a more significant number of tight binding antibodies in a more complex Ig-MS spectrum. The DoC may be determined from the peaks of the mass-domain spectrum through the use of Equation 2.

In some embodiments, a spectral correlation coefficient may be determined. The spectral correlation coefficiency may be determined from the peaks of the mass-domain spectrum through the use of Equation 3.

The proteoforms and complexes thereof identified by any of the methods disclosed herein may be utilized in the methods disclosed herein may be formulated as proteoform products, such as pharmaceutical compositions, that include: (a) an effective amount of one or more identified proteoforms or complexes thereof; and (b) one or more pharmaceutically acceptable carriers, excipients, diluents, stabilizers, preservatives, anticoagulants, and the like. Suitably the pharmaceutical composition may be an intravenous immunoglobulin (IvIG) product or a convalescent plasma product that may be prepared from one or more immunoglobulin donors.

As used herein, a “subject” may be interchangeable with “patient” or “individual” and means an animal, which may be a human or non-human animal. In some embodiments, the subject is a subject in need of treatment. A “subject in need of treatment” may include a subject having a disease, disorder, or condition that is responsive to therapy with a proteoform identified by the methods disclosed herein. For example, a “subject in need of treatment” may include a subject having an infection, such as a viral, bacterial, or fungal infection. In some embodiments, the subject is a subject that may need a treatment. A “subject that may need a treatment” may include a subject suspected of having a disease, disorder, or condition that may be result in the subjects generation of a proteoform or complex thereof indicative of the disease, disorder, or condition. For example, a “subject that may need a treatment” may include a subject that may have an infection, such as a viral, bacterial, or fungal infection, that is capable of inducing the generation of an immunoglobulin indicative of the infection.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the proteoform or complex thereof, upon single or multiple dose administration to the subject, which provides the desired effect. The disclosed methods may include administering an effective amount of the disclosed compounds (e.g., as present in a pharmaceutical composition) for treating an infection.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

IgG profiling may be performed on blood samples collected in PAXgene tubes from COVID-positive transplant recipients and control subjects (both with and without immunosuppression as described above). Collected blood samples will stored in at -80° C. until sample preparation and analysis. Samples will be fast thawed and centrifuged. There the serum containing IgG1-4 subtypes will be incubated with Protein A beads for IgG capture via immunoprecipitation (IP). The beads will be washed in a process optimized to avoid non-specific binding, and the isolated IgGs will be eluted using a defined process [Park, H.-M. Anal. Chem. 2020, 92, 2, 2186-2193]. The antibodies will be then cleaned using a 30 kDa cut-off membrane on a SampleStream platform (SS)and online submitted to individual ion mass spectrometry - I2MS [Kafader, J.O. Nat Methods 17, 391-394 (2020)]. Acquired raw data files will be processed to have charge assigned and the mass distribution of IgGs provided. The obtained spectra will be compared in between patients for direct monitoring of IgG responses and possible single antibody clone resolution. Data from this overall “IP-SS-I2MS” process will be subjected to statistical analysis using standard ANOVA, customized to the study designs enabled from biobanking low thousands of individual blood samples and de-identified patient metadata. Correlation to standard ELISA-based serological testing will enable identification of IgG subtypes.

Alternative embodiments may include IgG deglosylation using PNGaseF and/or reduction of disulfide bonds to liberate IgG heavy and light chains — both inside the SampleStream channel.

### Example 2

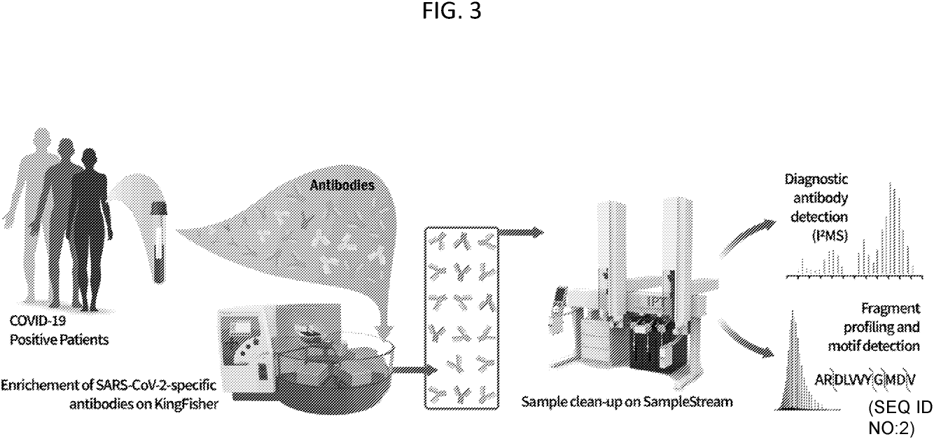

To enrich SARS-CoV-2-specific antibodies, magnetic beads may be conjugated to recombinant SARS-CoV-2 spike receptor binding domain (RBD) expressed in HEK-293T cells. Spike RBD plasmid may be used to make ~50 mg/L of competent spike RBD. [Amanat et al. Nature Medicine doi: 10.1038/s41591-020-0913-5; Stadlbauer et al. Curr Protoc Microbiol. 2020;57(1):e100. doi:10.1002/cpmc.100] After conjugation to beads, plasma may be incubated from COVID-19 recovered patients from the COVID-19 Convalescent BioBank and optimize wash conditions to remove non-specific binders or antibodies with very low affinity to the spike RBD. Optimization may be performed on the Thermo KingFisher (FIG. 3). Bound proteins may be eluted under various conditions and SDS-PAGE with staining with Coomassie may be used to determine which conditions provide the best recovery of antibodies.

### Example 3

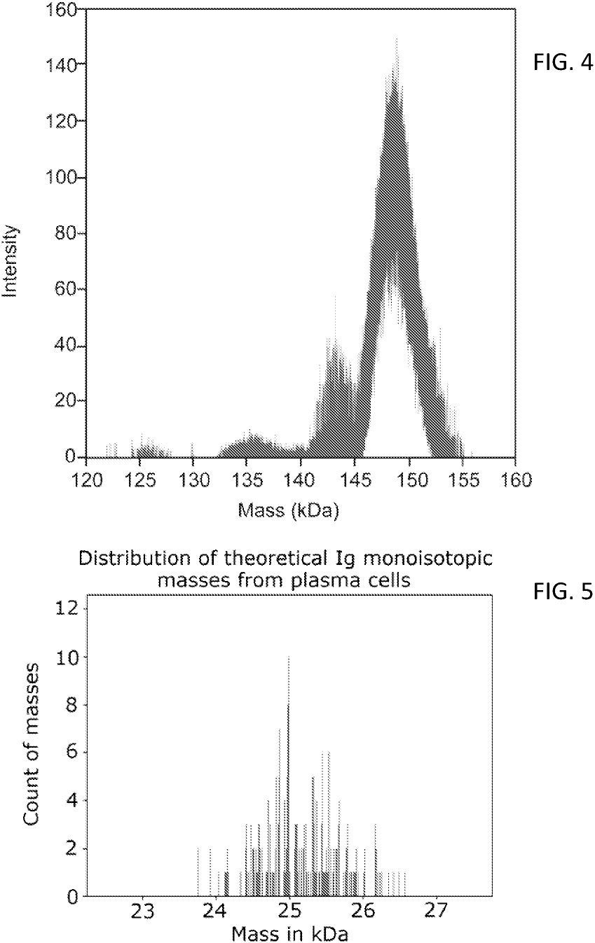

Experimental IgG profile data (FIG. 4) may be compared to theoretical a profile data (FIG. 5) inferred from heavy chain CDR1-3 sequences from the N-terminal domain of the IgG’s obtained from from B-cell sequences. Baeyesian or similar methods may be used to establish priors and use intact mass patterns to compare patients. This will allow for top-down mass spectrometry to gain sequence information for the entire variable regions of the heavy and lights chains.

### Example 4

Herein, we report a fundamentally new data type in human serology that provides a readout of all immunoglobulins produced against a specific antigen with molecular resolution using proteomics methods. IgMS is the first mass spectrometry-based platform capable of capturing the complete pattern of the light chain and heavy chains of immunoglobulins produced by the adaptive immune system. Specifically, we applied this new approach to COVID-19 convalescent individuals to track their immune response complexity against the RBD region of the spike protein from SARS-CoV-2. Explicitly, we generate quantitative patterns containing all variability from the 3 CDRs regions from light and heavy chains in a single analysis.

Here, we present Ig-MS, a novel serological readout that captures the immunoglobulin (Ig) repertoire at molecular resolution, including entire variable regions in Ig light and heavy chains. Ig-MS uses new advances in protein mass spectrometry (MS) for multi-parametric readout of antibodies, with new metrics like Ion Titer (IT) and degree of clonality (DoC) capturing the heterogeneity and relative abundance of individual clones without sequencing of B cells. We apply Ig-MS to plasma from subjects with severe and mild COVID-19, using the receptor-binding domain (RBD) of the spike protein of SARS-CoV-2 as the bait for antibody capture. Importantly, we report a new data type for human serology, with compatibility to any recombinant antigen to gauge our immune responses to vaccination, pathogens, or autoimmune disorders.

Intact proteins have many proteoforms¹⁰, and in the case of antibody heterogeneity this could be captured if there was a molecular readout with enough resolution and specificity. Past efforts have been able to detect monogammapathies (e.g., B cell cancers like multiple myeloma), but a direct readout of Ig repertoire at high resolution is not currently possible¹¹. Addressing this, we established Ig-MS, which isolates antibodies against a specific target from the plasma of patients and controllably breaks them down into their heavy and light chains (HC and LC). Once created, HC and LC fragments are analyzed by individual ion mass spectrometry (I²MS), a breakthrough approach that generates mass distributions for extremely heterogeneous protein samples using ~500 fold more dilute samples than classical protein MS analysis¹². Using one of the two workflows (FIG. 7A), Ig-MS provides compositional profiles for Ig repertoires, their degree of clonality, and titers, including for an initial cohort of COVID-19 subjects. With increased resolution for molecular serology, Ig-MS provides high-value clinical correlates of viral neutralization, the presence, extent, and course of the disease, and/or assess the degree of protection after vaccination.

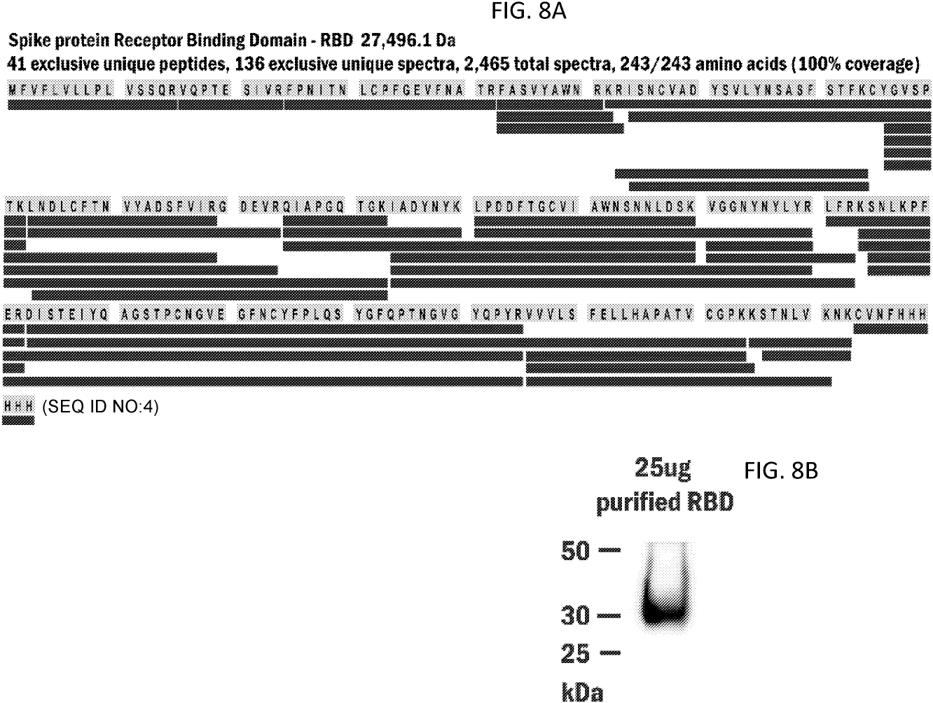

Benchmarking the Ig-MS Assay. The RBD of the spike protein from SARS-CoV-2 (Wuhan strain) was expressed, purified from HEK293 cells, and characterized by both bottom-up proteomics (100% sequence coverage) and western blot (FIGS. 8A-8B). Purified RBD was covalently attached to magnetic beads and used as bait for binding and enriching antibodies against SARS-CoV-2-Spike-RBD (Ig-RBD) from the plasma of COVID-19 convalescent individuals (FIG. 1A). One hundred nanograms of a standard mAb CR3022 was used as an internal standard in all Ig-MS.

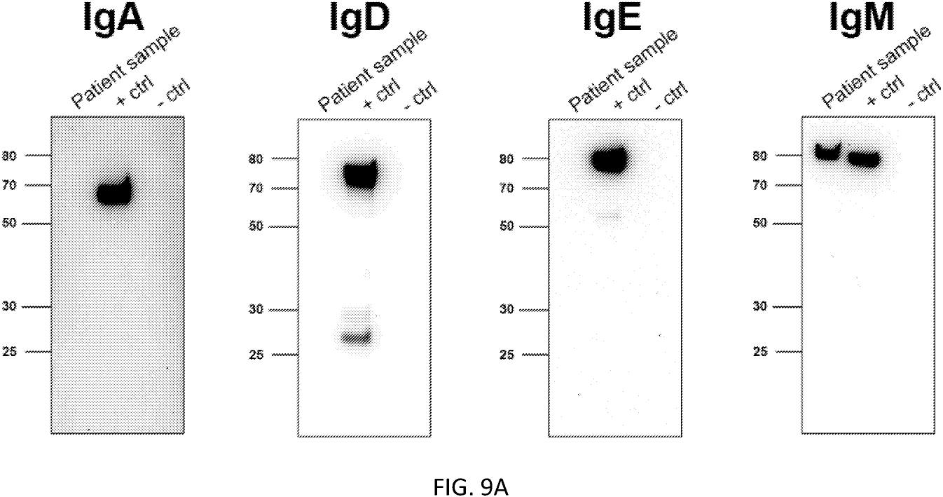

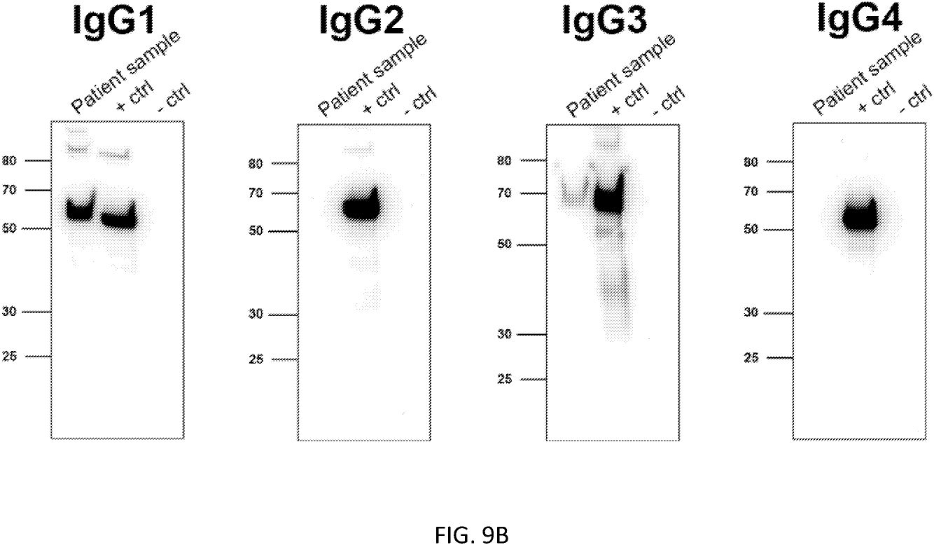

Proof-of-concept experiments used samples derived from a single COVID-19 convalescent individual featuring a high ELISA titer of Ig-RBD, hereafter referred to as P1877. Enrichments from plasma derived from P1877 were performed to optimize parameters such as covalent bead-loading chemistry, the wash, and elution buffer conditions to increase capture and suppress non-specific binding. As shown in FIG. 7B, the optimized pull-down protocol for 100 uL of plasma efficiently captured Ig-RBD, which was eluted to >90% in the optimized elution conditions. Pull-downs from P1877 were used to profile the antibody isotypes (IgA, IgD, IgE, and IgM) and IgG subclasses (IgG1, IgG2, IgG3, and IgG4) being enriched. A specific western blot revealed that IgG1, IgG3, and IgM were the primary isotypes isolated (FIGS. 9A-9B), similar to what has been reported in the literature¹³.

Following antibody enrichment, two distinct workflows were developed to prepare isolated Ig-RBD for Ig-MS analysis (FIG. 7A). In workflow 1, Ig-RBDs were eluted intact, combined with 100 ng of standard mAb CR3022, and fully reduced/denatured in the presence of a chaotropic agent to liberate HC (48-55 kDa) and LC (22-25 kDa) proteoforms. In workflow 2, patient Ig-RBD were digested with IdeS protease while still attached to the beads, generating F(ab′)₂ and Fc. In workflow 2, the protease and Fc species (containing HC glycosylation) are washed away. Eluted F(ab′) ₂ were denatured and reduced to yield the LC and the Fd domain (~25-28 kDa), which contains the N-terminal 220 amino acids of the HC and therefore captures the composition of all three Complementarity-Determining Regions (CDRs). Before the I²MS analysis step of Ig-MS, 100 ng of digested and reduced mAb CR3022 were spiked into the sample.

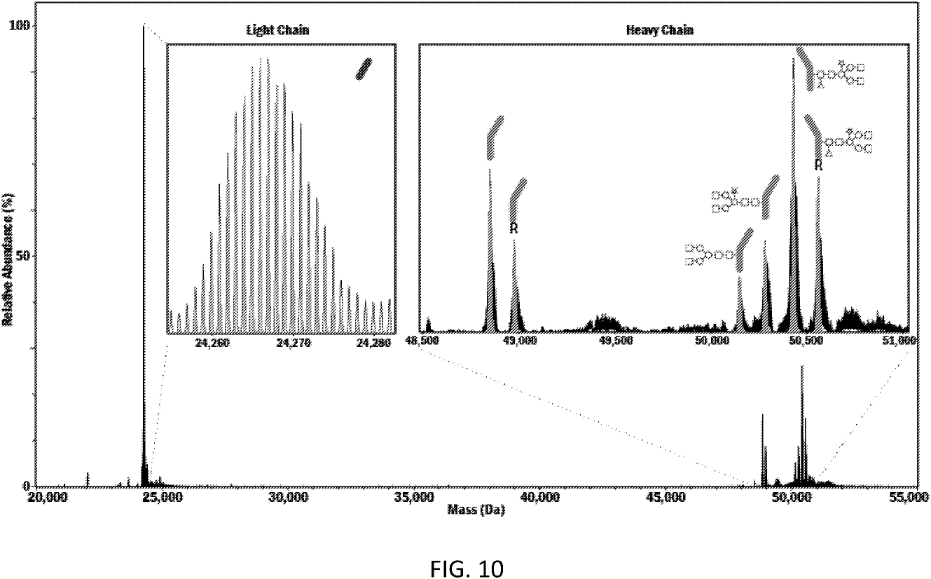

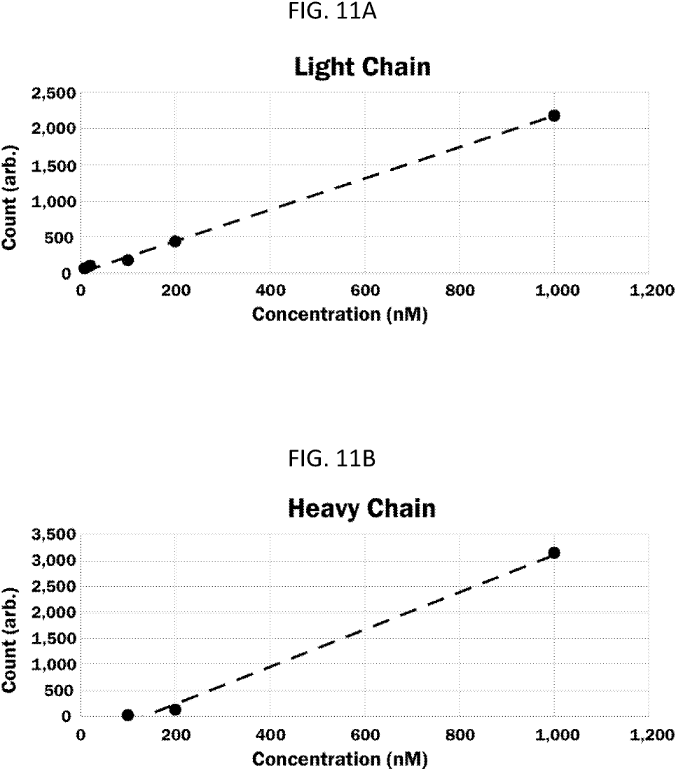

To benchmark Ig-MS, we first analyzed mAb CR3022 and annotated the LC and HC proteoforms (FIG. 10). Subsequently, we estimated the Ig-MS Limit of Detection (LOD) for the HC of NIST mAb (with glycosylation) at 100 nM, whereas the LOD for the LC was well below 10 nM (FIGS. 11A-11B).

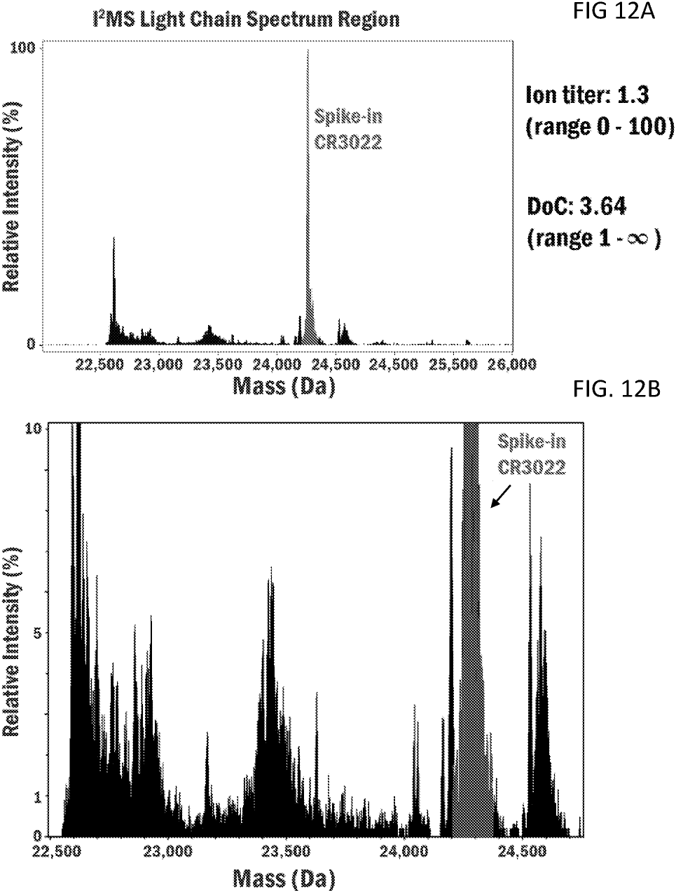

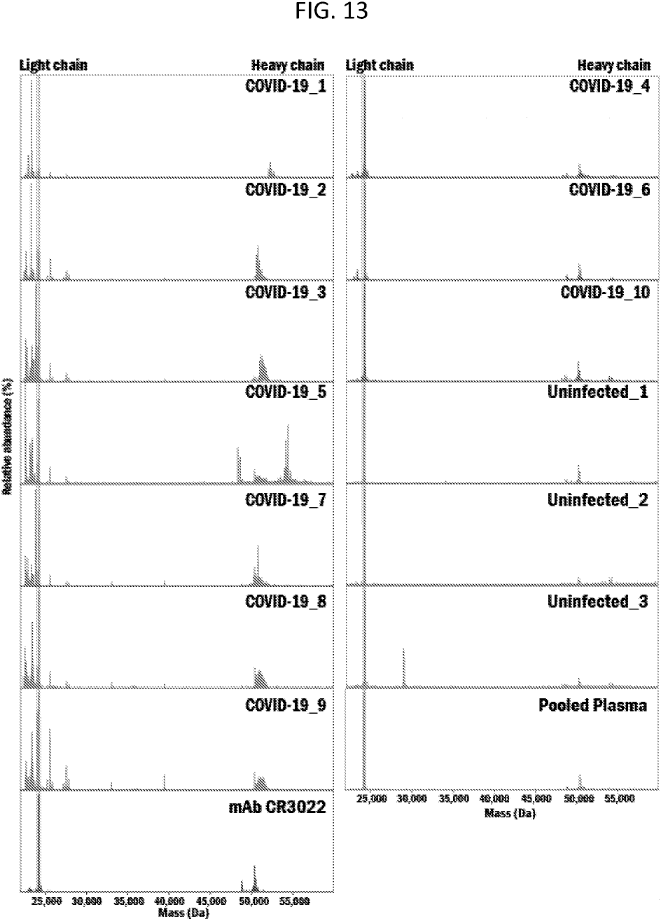

Next, we performed Ig-MS using workflow 1 on P1877, and FIGS. 12A-12B shows the spectrum obtained for the LC region. Like in FIG. 7C, the highlighted peak at ~24,268 Da represents the mAb CR3022 standard used to calculate Ig-MS metrics, as outlined in Equations 1-3. The other peaks with lower mass than the standard LC, ranging from ~22 to 24 kDa, are distinct LC proteoforms originating from B cell clones with different or isobaric CDR sequences in their variable regions. With this first glimpse at an Ig repertoire at the LC and HC levels, the presence of distinct proteoforms shows that single clone resolution is possible, with some present at high titer (i.e., ~500 ng/100 uL plasma). The current dynamic range for detecting different clones that evolve after VDJ recombination is approximately 100 (FIGS. 12A-12B).

Given that Ig-MS produced a new data type, we created two new metrics. The Ion Titer (IT) is similar to an ELISA titer and uses the intensity of the LC of the standard mAb CR3022 as a reference, and combines intensity of all other LC peaks and ranges from 0 to 100 (Equation 1). The second is the Degree of Clonality (DoC), a measure of the complexity of all LC clones (proteoforms) in the mixture and ranges from 1 to infinity, with higher numbers reflecting the presence of a more significant number of tight binding antibodies in a more complex Ig-MS spectrum. For the P1877, the calculated IT was 1.30, and the DoC was 3.64. We next sought to compare these IgG patterns and new metrics across patients and perform initial correlations with other COVID-19 antibody tests.

Ig-MS of an initial cohort. We deployed Ig-MS workflows 1 and 2 to survey the Ig population reactive to RBD in a cohort of seven hospitalized patients with severe COVID-19, three outpatients with mild COVID-19 disease, and three healthy people who never had COVID-19 (Table 1). The standard mAb CR3022 and commercial pooled plasma acquired before November 2019 served as positive and negative controls, respectively.

TABLE 1

Correlation of Ig-MS metrics with patient metadata. Average titers were determined with ELISA-based serological assays and surrogate virus neutralization titers from 10 COVID-19 convalescent patients. IgMS metrics for workflow 1 and workflow 2. Degree of clonality (DoC)

| Metadata | | | Titers | | | Workflow 1 | | Workflow 2 | |
|---|---|---|---|---|---|---|---|---|---|
| Patient | Hospitalized | Days from test to blood draw | ELISA | Promega | Surrogate Neutralization | Ion Titer | DoC | Ion Titer | DoC |
| COVID-19_1 | Yes | 20 | 1.78 | 50345 | 0.20 | 30.11 | 2.36 | 5.95 | 1.83 |
| COVID-19_2 | Yes | 10 | 2.19 | 179569 | 0.14 | 10.99 | 2.82 | 2.94 | 2.71 |
| COVID-19_3 | Yes | 27 | 2.16 | 98789 | 0.16 | 14.87 | 7.25 | 4.73 | 6.28 |
| COVID-19_4 | No | 90 | 0.66 | 2471 | 1.27 | 0.83 | 5.84 | 0.46 | 5.76 |
| COVID-19_5 | Yes | 57 | 2.16 | 73400 | 0.13 | 6.51 | 4.22 | 1.80 | 3.26 |
| COVID-19_6 | No | 50 | 0.58 | 843 | 1.86 | 0.65 | 3.08 | 0.35 | 4.14 |
| COVID-19_7 | Yes | 67 | 1.71 | 98706 | 0.11 | 6.22 | 6.03 | 1.64 | 3.07 |
| COVID-19_8 | Yes | 71 | 1.68 | 47852 | 0.14 | 6.30 | 3.67 | 2.03 | 3.14 |
| COVID-19_9 | Yes | 60 | 2.02 | 95191 | 0.13 | 8.59 | 7.01 | 2.37 | 4.34 |
| COVID-19_10 | No | Not tested | 0.47 | 704 | 1.70 | 0.94 | 4.41 | 0.34 | 5.76 |
| Uninfected_1 | - | - | - | - | - | 0.22 | NA | 0.28 | NA |
| Uninfected_2 | - | - | - | - | - | 0.39 | NA | 0.37 | NA |
| Uninfected_3 | - | - | - | - | - | 0.38 | NA | 0.55 | NA |
| Standard mAb | - | - | - | - | - | 0.55 | NA | 0.14 | NA |

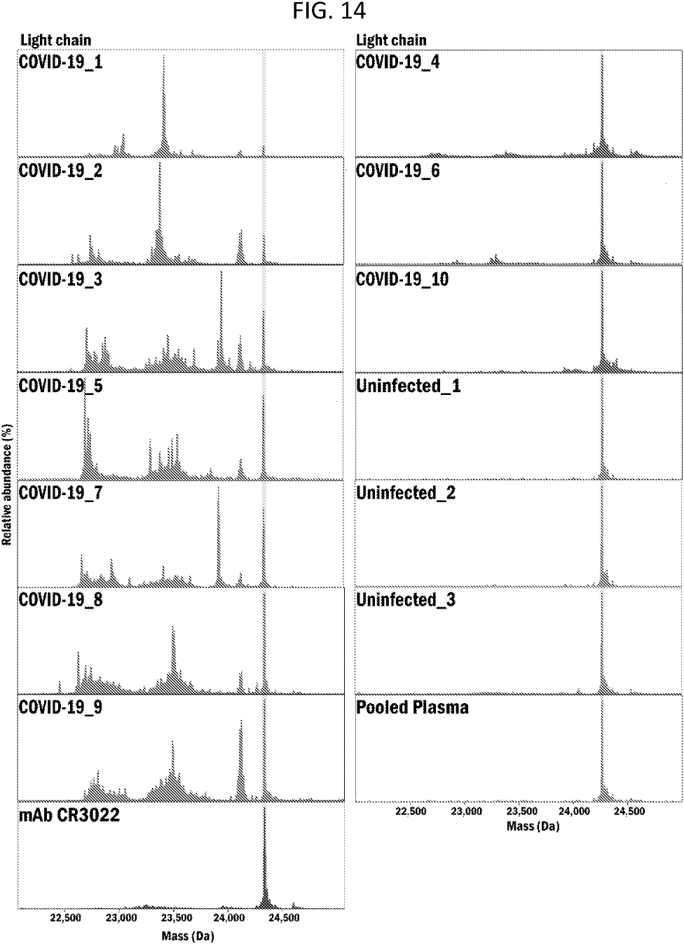

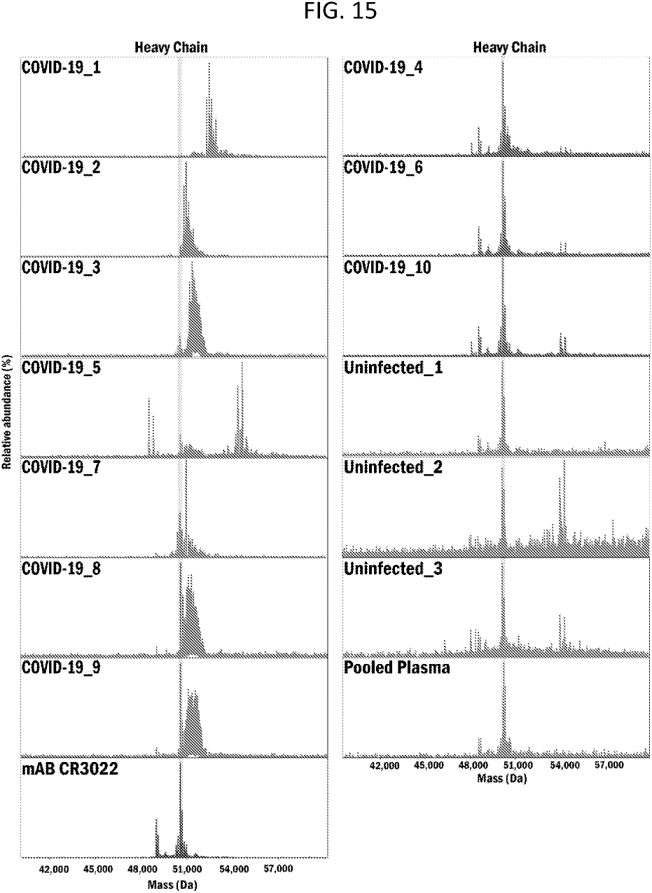

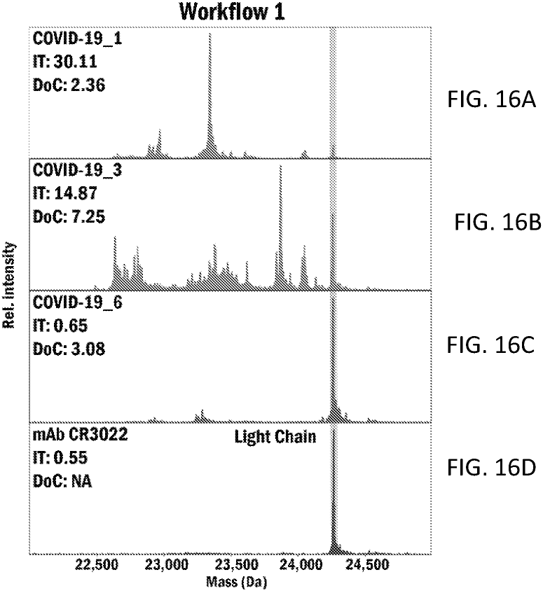

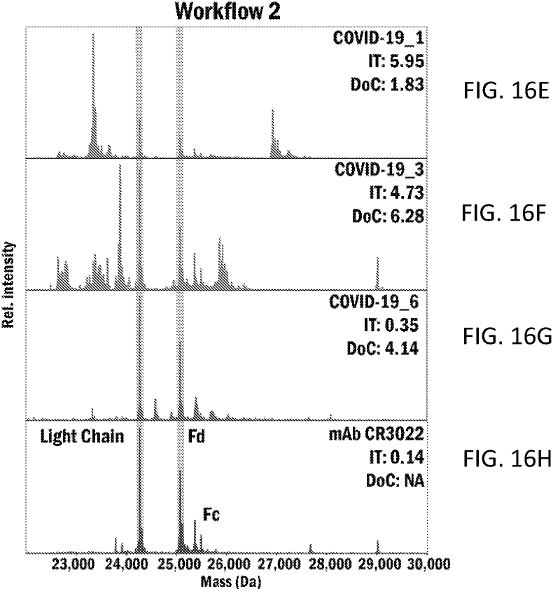

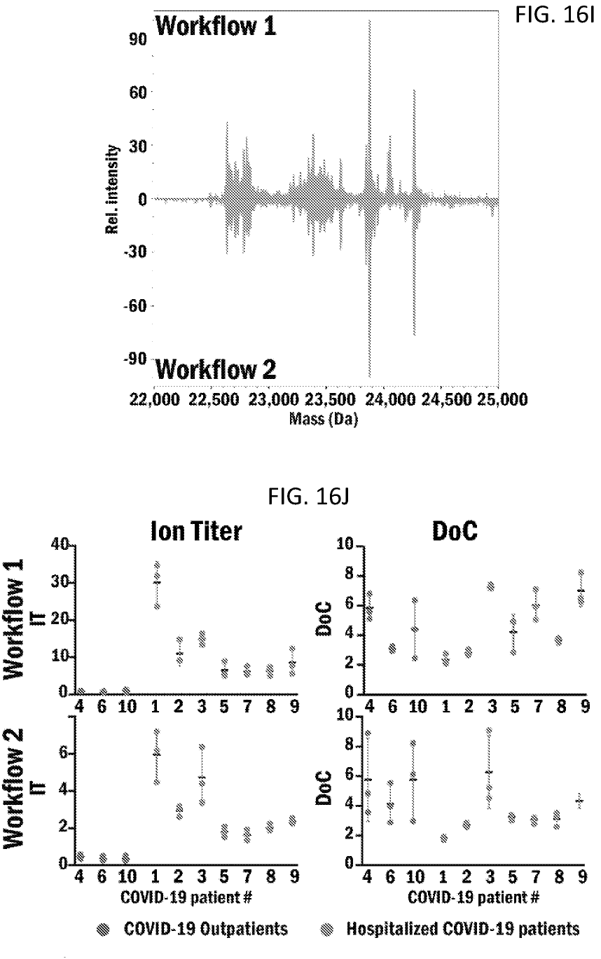

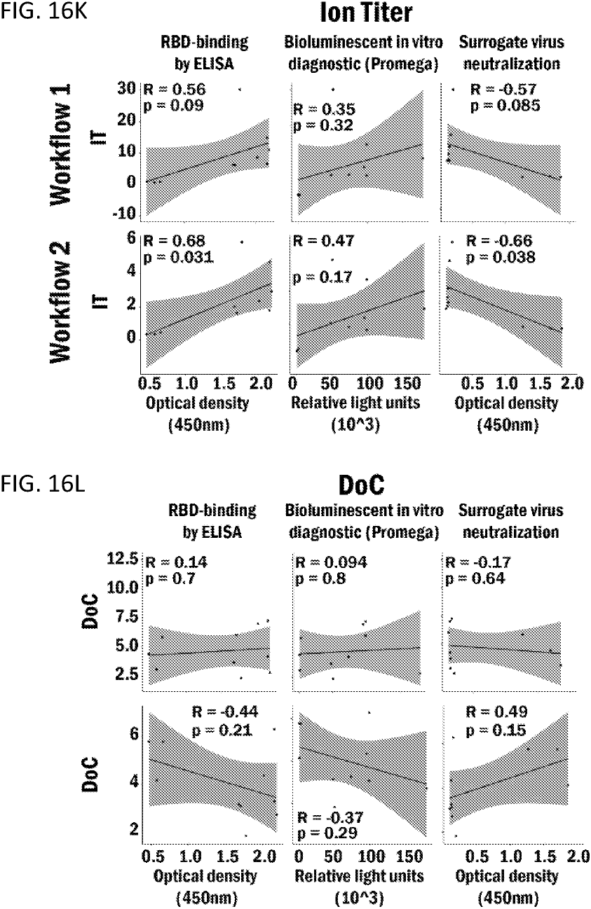

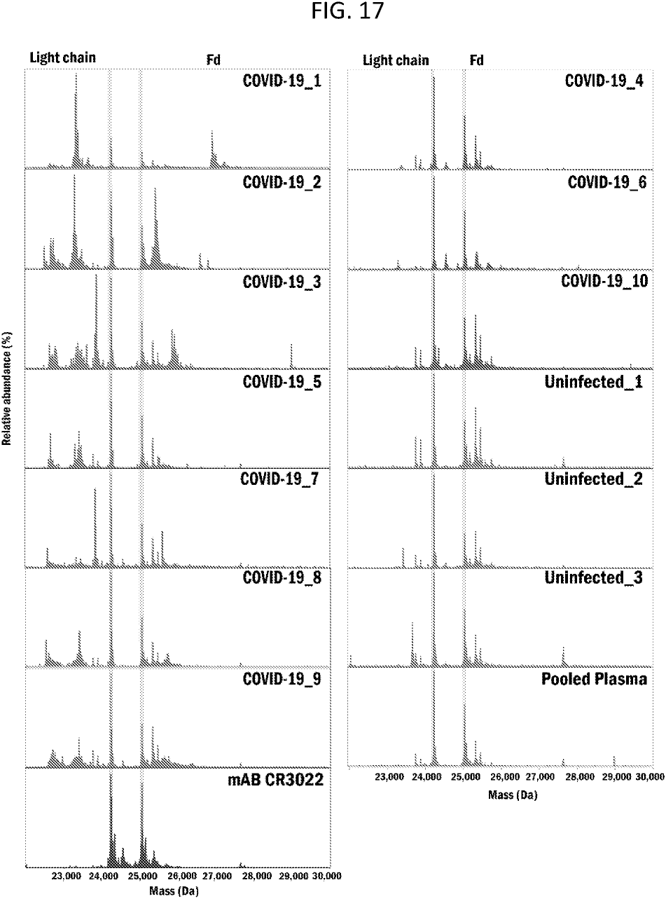

From workflow 1, the HCs and LCs are all shown in FIG. 13 for this cohort, while FIGS. 14-15 present expansion of LC and HC regions, respectively. FIGS. 16A-16L highlights the LC region for examples of three general groups of immunological responses: (1) high IT (30.11) and low DoC (2.36) as observed in one of the hospitalized patients (COVID-19_1) (FIG. 16A); (2) moderately high IT (14.87) and high DoC (7.25) as observed on the hospitalized patient COVID-19_3 (FIG. 16B); and (3) low IT (0.65) and low DoC (3.08) as observed on the outpatient COVID-19_6 (FIG. 16C).

The same 13 samples were processed using workflow 2, which generated the LC and Fd for readout by Ig-MS. FIG. 17 exhibits the obtained spectra for all samples. The spectra from patients COVID-19_1 (FIG. 16E), COVID-19_3 (FIG. 16F), and COVID-19_6 (FIG. 16G) displayed the same immunological response pattern as those obtained with workflow 1, showing the self-consistency of the two workflows.

Distinct proteoform masses representing all the LCs and Fds were clearly discernable in both workflows (FIGS. 16A-16H). The LC proteoforms have a mass range between 22-25 kDa (FIGS. 16A-16H, to the left side of the standard LC highlighted in gray) and comprise the entire length of the LC, including the three CDRs (VL) and conserved region (CL). We can identify κ and λ LCs in the Ig-MS spectra based on the amino acid differences in the conserved regions. In FIG. 16B, the masses between 22.5 to 23 kDa belong to lambda LCs and those from 23 to 24 kDa to kappa LCs¹⁴. A correlation coefficient (Equation 3) was calculated and showed an average value of 0.95 for technical replicates. The same basic patterns of LC proteoforms were observed with both workflows applied to the same sample. For example, a comparison of LC proteoform patterns for patient COVID-19_3 showed a correlation coefficient of 0.77 (FIG. 16I). FIG. 16J displays the IT and DoC values for the subject’s samples. The IT values are significantly higher in hospitalized patients for both workflows (workflow1: F=12.64, 2 and 33 DF, p<0.001 and workflow 2: F=18.26, 2 and 33 DF, p<0.001) compared to outpatients, a result corroborated with standard serological tests from previous studies^{15,16}. No statistically significant differences were observed for the DoC metric in these groups.

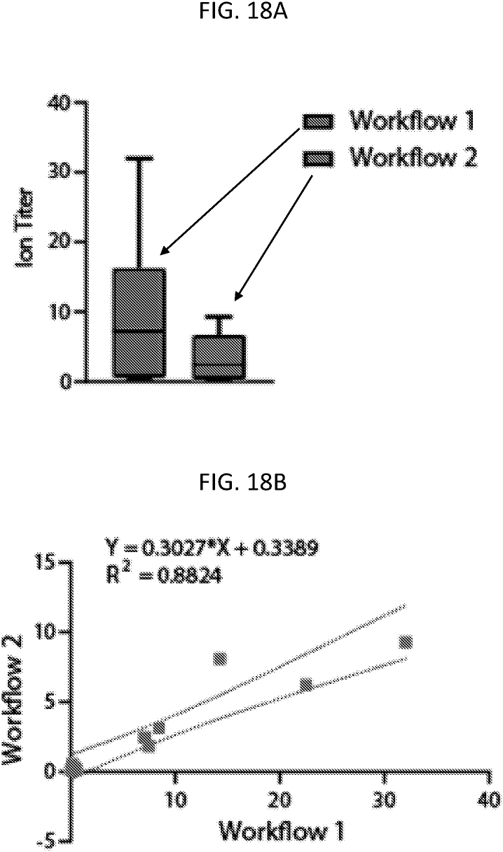

Ion Titers were also calculated exclusively for LC regions and compared between workflow 1 and 2. Although differences occur in the range of ion titers observed between the two workflows, there was a strong correlation (R² = 0.88) between them, indicating a systematic bias (FIGS. 18A-18B). We believe that this bias arises from the differences in sample and standard preparation. However, the average cross-correlation of the LC from the two studies is 0.78 ±0.04, indicating that the ratios among peaks and the relative proteoform amounts are conserved in both workflows of Ig-MS.

With workflow 2, we can analyze Fd proteoforms that include three CDRs from the HC (VH) and the constant region CH1 (FIGS. 16A-16L, to the right side of the standard Fd highlighted in gray at workflow 2) besides all possible individual variation in the constant region. This workflow captures the post-VDJ sequence recombination and dissects the Fc glycosylation pattern away from the intact HC observed on workflow 1, reducing the detected complexity. Across this initial study, it appears to be a more considerable degree of heterogeneity in the Fd relative to the LC from the same person.

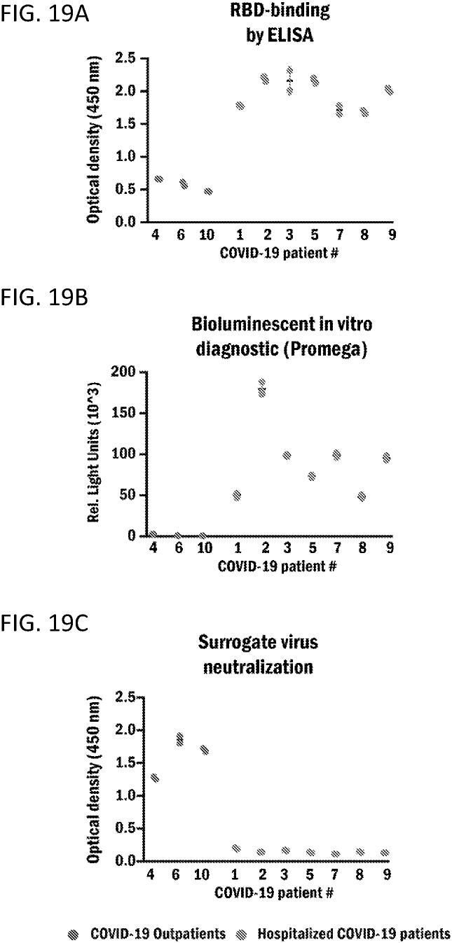

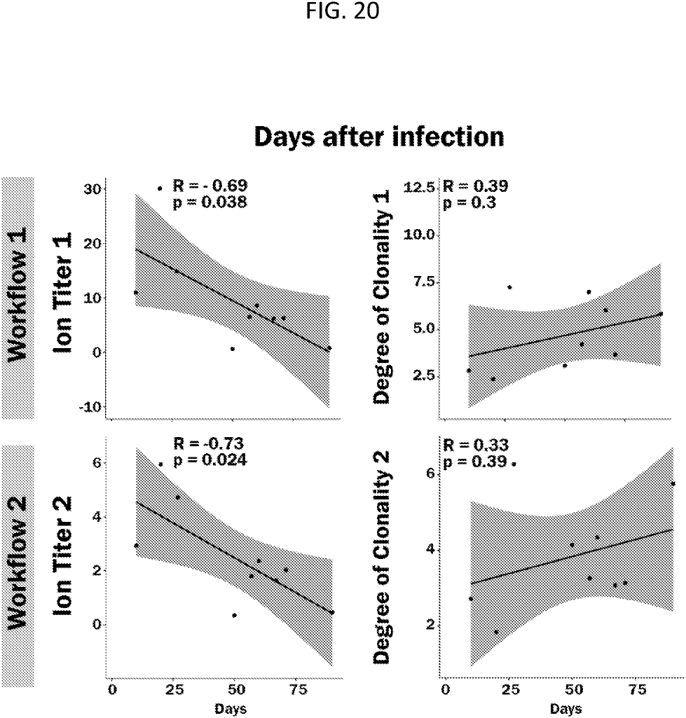

We next probed the correlation of ITs obtained with the two Ig-MS workflows with titers of anti-RBD antibodies quantified with ELISA¹⁷ and a commercial in vitro diagnostic using bioluminescence. We also determined the correlation between IT and neutralization efficiency obtained with a Spike-specific pseudovirus neutralization test (FIGS. 16K-16L, FIGS. 19A-19C, and Table 1). Pearson’s correlation (R) (FIG. 16K) indicated a significant positive correlation of IT obtained with workflow 2 and the ELISA titers (R=0.68) and a negative correlation with surrogate neutralization (R=-0.66). The same tendency was observed for ITs determined with workflow 1 (R>0.55) but with p-values higher than 0.05. The correlation analysis of the ITs of both workflows with the bioluminescence was low (R≤0.47) and not significant. Furthermore, DoCs from both workflows did not correlate with the others assays (FIG. 16L), indicating that the number of different Ig signals produced by Ig-MS did not show a correlation with neutralization potential or overall titer in this initial study. For both workflows, there was a significant negative correlation between ITs and days after infection (FIG. 20). These results suggest that there is a reduction in the total amount of antibodies over time, but the general degree of complexity in the immune response does not change. Additionally, virus neutralization is more related to the total amount of antibodies in the plasma than to clonal heterogeneity in the Ig repertoire.

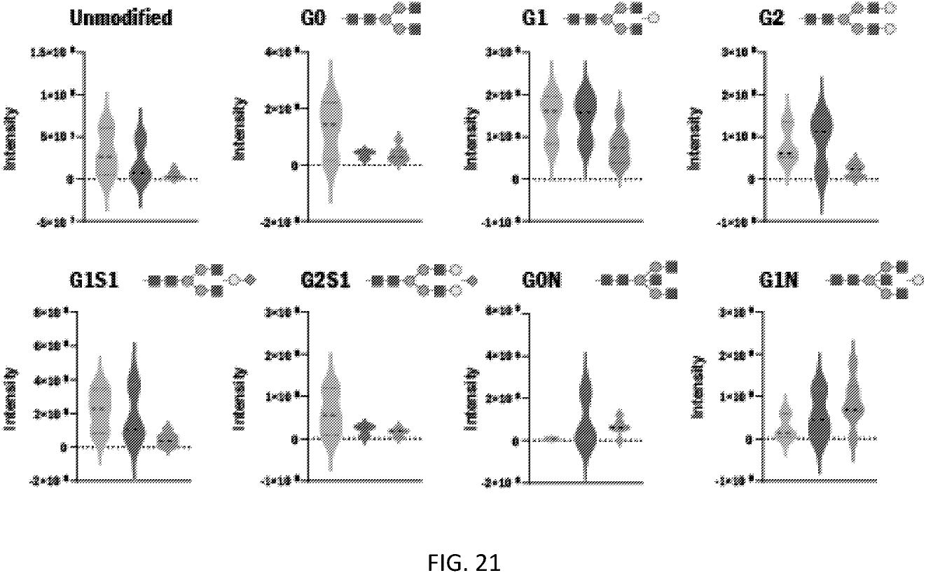

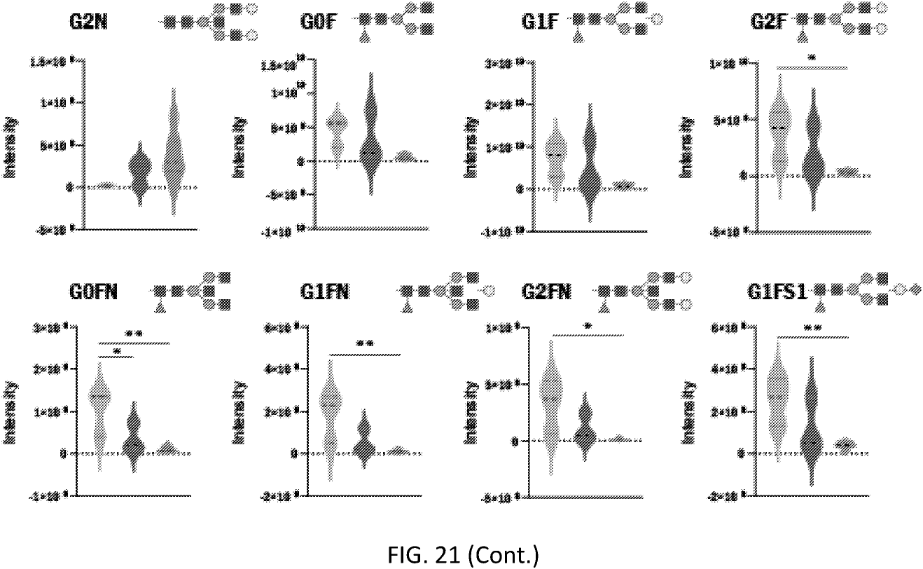

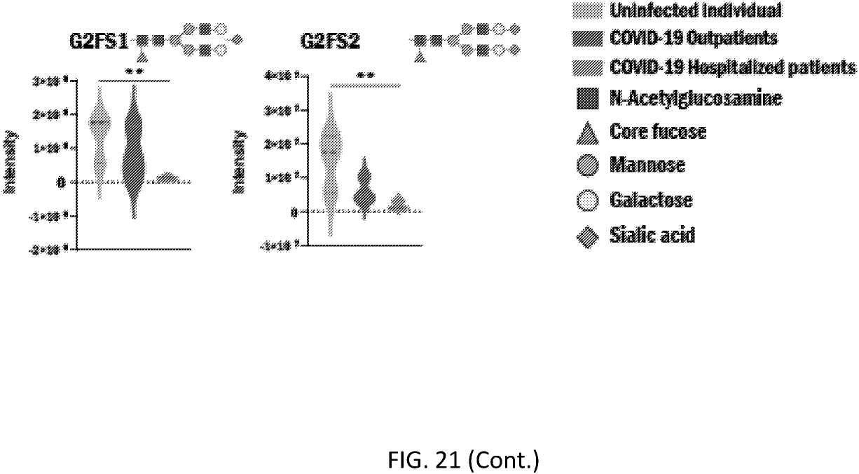

IgG glycosylation analysis. N-glycosylation at position 297 of the HC mediates the antibody’s effector functions. Further, the glycan moieties are highly variable and functionally relevant. To explore the IgG glycosylation pattern of our three study groups, we used bottom-up proteomics to quantify 17 different glycan structures (attached to the IgG peptide) and the unmodified peptide¹⁸. A Benjamini and Hochberg correct ANOVA analysis on the results from the three groups indicated that seven fucosylated glycans (G2F, G0FN, G1FN, G2FN, G1FS1, G2FS1, and G2FS2) were differentially regulated. A second one-way ANOVA with Tukey’s multiple comparison test revealed that those fucosylated glycans were down-regulated in the hospitalized group compared to the uninfected group (FIG. 21).

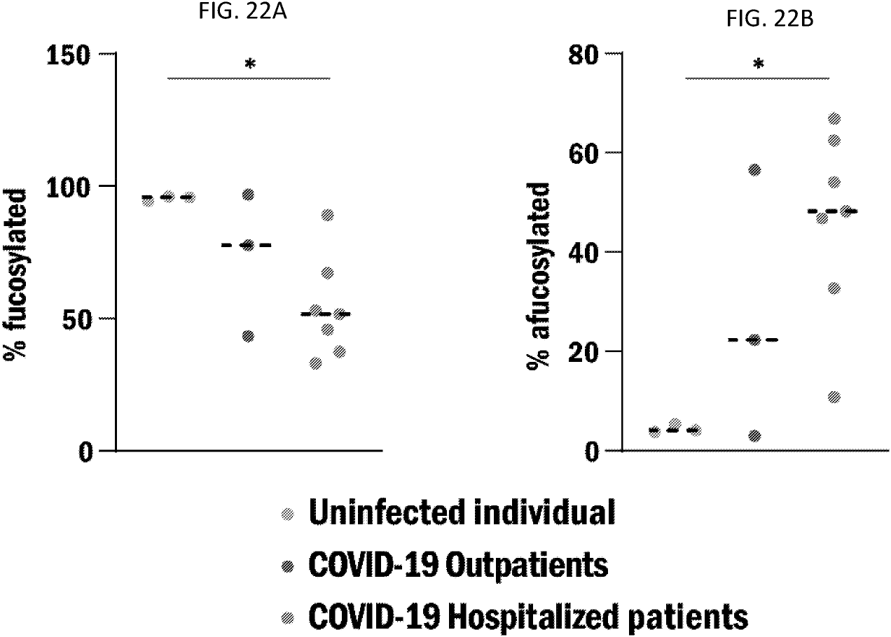

Additionally, the average percent of the total fucosylated glycans was 53.9% ±17.7% for the hospitalized group and 95.5% ±0.8% for the uninfected individuals (FIGS. 22A-22B). Similar results were previously reported¹⁹, and afucosylated IgG induces increased antibody-dependent cellular cytotoxicity by rising IgG-Fc receptor IIIa (FcγRIIIa) affinity²⁰.

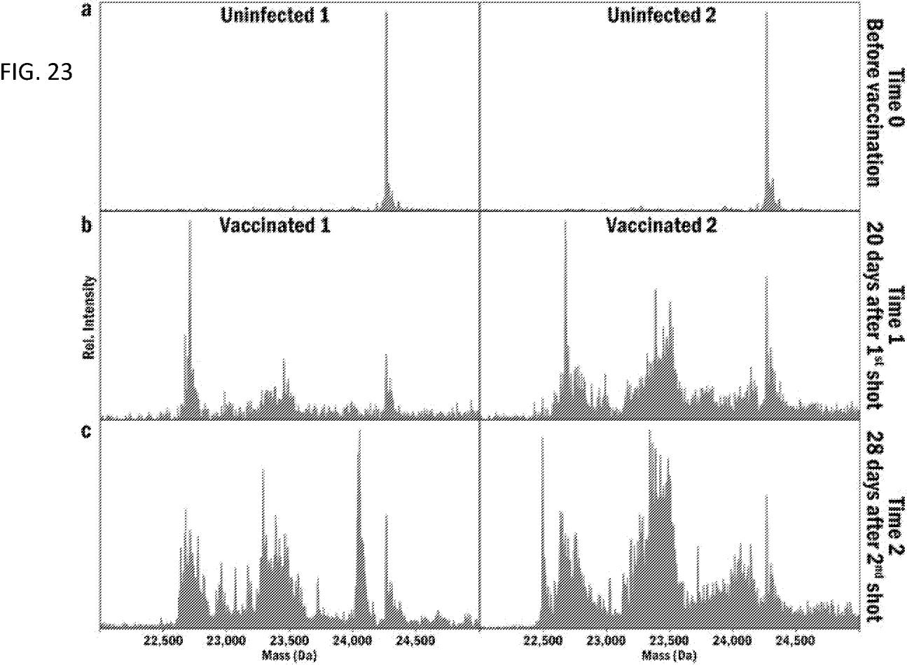

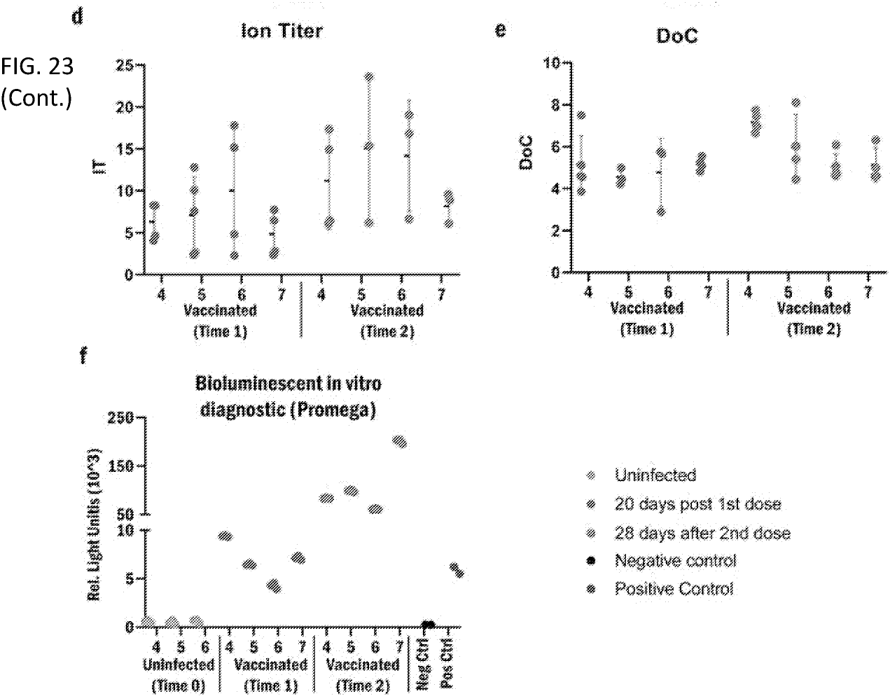

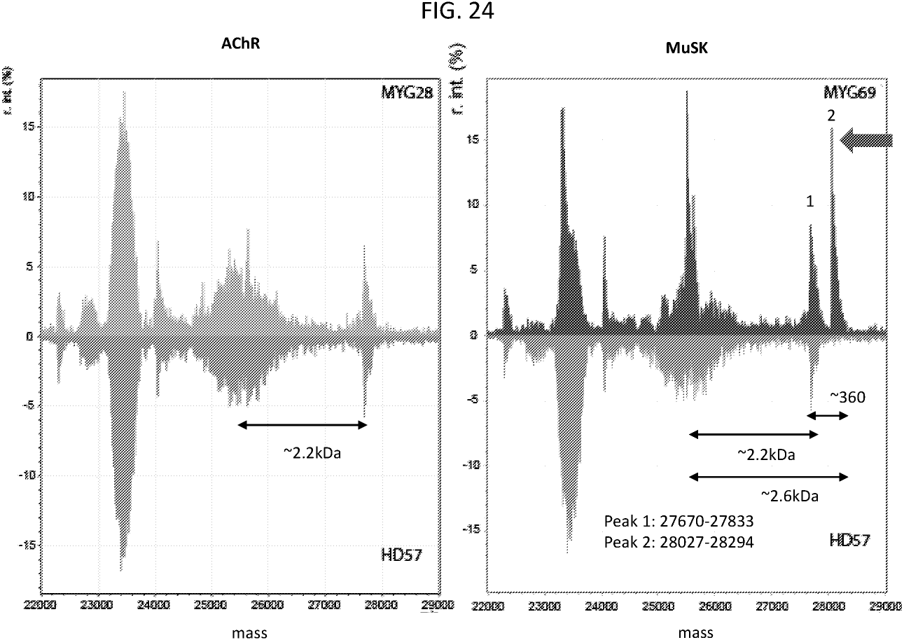

Vaccination analysis. FIG. 23 shows Ig-MS readouts as well as IT and DoC from workflow 1 on the vaccinated cohort. Comparision to the bioluminescent in vitiro diagnostic is also provided.

### Discussion

Antibody titers can be used to indicate the extent of immunity and disease severity for COVID-19^{15,16}. Ig-MS results showed that IT from the two workflows were self-consistent and correlated with traditional colorimetric/fluorimetric tests and a surrogate neutralization assay. The DoC metric and patterns of responses did not correlate with these assays, yet their variance in the human population needs to be determined. We observed significant differences in hospitalized from outpatients and reduced IT overtime of convalescence, similar to previous reports²¹. With this initial report, Ig-MS will function, prompting three general use cases: 1) provide a new, multi-parametric correlate of protection, 2) as a diagnostic to indicate the course of disease, and 3) striating lots of convalescent plasma to quantify protective potential and better control plasma collection campaigns²². Additionally, antibody amounts and clonal variation can play a complementary role in vaccine campaigns that can strongly correlate to protective immunity (i.e., provide a reliable surrogate to neutralizing titers).

Correlation with B cell Sequencing. Reports in the literature have begun to quantify the extent of convergence in the sequences of antibodies by deep sequencing of B cell receptors^{23,24}. They demonstrated a consistency of the stereotypical immune response, and it is a great resource to identify potential therapeutic and prophylactic antibodies. Such convergence can also be detected by Ig-MS because it provides intact mass patterns that reflect the combination of LC and HC CDRs. This is unlike approaches using tryptic digestion of antibodies²⁵⁻²⁹. Indeed, no technology can sequence the whole antibodies directly, but high sequencing coverage of LC and HC from monoclonal antibodies is possible today³⁰.

Future of COVID-19 Testing with Ig-MS. Importantly, Ig-MS is adaptable just by switching the affinity resin, so use on RBD variants of Spike protein in virus strains (like the B.1.1.3 and P.1) will probe vaccination effectiveness and Ig-MS utility as a new correlate of protection in serology. In addition, there are reports of neutralizing epitopes in the N-terminal Domain (NTD) of Spike, and we can further probe this elicited antibody repertoires³¹. Analysis of longitudinal samples from individuals infected with SARS-CoV-2 and after vaccination will track the immune response at high resolution^{21,32}. Ig-MS should also be applicable to IgA’s, IgMs, and other isotypes of immunoglobulins in the adaptive immune response.

In summary, we report a new and unique data type for human serology, using COVID-19 cases as the first example. Until now, no serological test was capable of accessing the relative abundance of each antibody generated against a specific antigen. Ig-MS is the first method capable of accessing both information simultaneously using the specificity of a fundamental advance in MS of individual protein ion¹² to create a unique display of the IgG repertoire of a human being at molecular resolution. Ig-MS successfully captured the clone populations of RBD-reactive immunoglobulins and showed preliminary solid data on a limited cohort of COVID-19 patients. In the future, a more automated form of Ig-MS will address larger cohorts, use less sample, and extensively sequence CDR variable regions for comparison with methods for single B cell sequencing, like Ig-seq³³.

Patient Cohorts and Plasma Sampling. Throughout this work, plasma from convalescent COVID-19 donors and those in control groups were collected under IRB NUMBER 00000482 by the clinical team at Rush Hospital. Patients were sampled post-infection by at least 10 days after the symptoms started. Plasma was isolated from the blood through centrifugation at 1,500 × g for 10 min. Plasma from a convalescent patient featuring a high titer of anti-SARS-CoV-2-RBD antibodies was purchased from AllCells (patient ID #3041877) and used as a standard positive control throughout the study. As a negative control/blank background, pooled plasma collected before the emergence of the SARS-CoV-2 pandemic was used (Fisher Scientific BP2657100 UNSPSC 12352207, purchased May 05, 2019).

RBD-binding by ELISA. Hisorb Ni+ plates (Qiagen) were coated with 100 µL of His-tagged RBD (BEI) at a concentration of 2 µg/mL overnight at 4° C. Plates were blocked 100 µl per well of 3% non-fat milk prepared in PBS with 0.1% Tween 20 (PBST) was added to the plates at room temperature for 1 hr. Next, heat-inactivated plasma from COVID-19 patients was diluted 1:10 in PBS and added at 100 µL per well for 2 hr at RT. Plates were washed with PBS-T 3 times, followed by incubation with secondary anti-human IgG Fc HRP (1:4000) for 1 h. Plates were washed 3 times with PBS-T followed by the addition of TMB substrate for 10 min. The reaction was stopped using 3 M HCl and read at OD 450 nm on the Biotek Cytation 3.

SARS-CoV-2 Surrogate Virus Neutralization Assay. COVID-19 patient plasma samples were heat-inactivated at 56° C. for one hour. Following heat inactivation, samples were diluted at a volume ratio of 1:9 in sample dilution buffer and mixed with a 1:1000 HRP-conjugated RBD solution in HRP dilution buffer at a volume ratio of 1:1 and incubated at 37° C. for 30 minutes. Following incubation, samples and kit-provided controls were added to an ACE2-coated 96-well plate, 100 µL/ well in duplicate, and incubated at 37° C. for 15 minutes. The plate was then washed four times with 1X wash solution and incubated with 100 µL/well TMB solution in the dark at room temperature for 15 minutes, followed by the addition of 50 µL/well reaction stop solution. The absorbance was then read on a biotek Cytation 3 plate reader at 450 nm. The protocol is adapted from GenScript SARS-CoV-2 Surrogate Virus Neutralization Test Kit (Cat. No. L00847-A), and all reagents used were provided in the test kit.

Recombinant Monoclonal Antibody. Human recombinant monoclonal Anti-SARS-CoV-2 antibody, mAb CR3022 produced in Nicotiana bethamiana (tobacco plant) was obtained from Novici Biotech LLC (Lot NCV _051520B) as a 1.0 mg/mL solution in PBS (pH 7.2). This reagent binds RBD of the Spike protein from SARS-CoV-2 and is also available through BEI Resources, catalog number NR-53876. The recombinant mAb was stored at 4° C. prior to use.

Recombinant expression of SARS-CoV-2 spike protein receptor-binding (RBD) domain. The plasmid pCAGGS SARS-CoV-2 RBD comprises an N-terminal signal sequence, amino acids 319-541 of the spike protein from SARS-CoV-2 (the receptor-binding domain, RBD), and a C-terminal 6-His tag³⁴. This vector was obtained from BEIResources (BEINR-52309) and expressed recombinantly using the Expi293 Expression System as follows: Expi293F™ cell culture (1 L total) was maintained in a 37° C. incubator with ≥80% relative humidity, 8% CO₂ on an orbital shaker platform and sub-cultured at cell density 3-5 × 10⁶ viable cells/mL. One day before transfection the cells were seeded to a final density of 2.5 × 10⁶ viable cells/mL and allowed to grow overnight. On the day of transfection, the culture was diluted to 3 × 10⁶ viable cells/mL with fresh, prewarmed Expi293™ Expression Medium. The transfection was performed using ExpiFectamine™ 293 Transfection Kit. Briefly, DNA/Opti-MEM™ I and ExpiFectamine™ 293/Opti-MEM™ I mixtures were prepared separately and incubated at room temperature for 5 min. These mixtures were then combined, and the total complexation mixture was incubated at room temperature for another 20 min. after which it was slowly mixed into the cell culture to initiate transfection. 18 h post-transfection ExpiFectamine™ 293 Transfection Enhancer 1 and ExpiFectamine™ 293 Transfection Enhancer 2 were added to the culture. The cell culture supernatant was collected after 6 days and prepared for purification.

Purification of SARS-CoV-2 RBD: Purification was performed on AKTAxpress (GE Healthcare Life Science) FPLC purification system. Clarified cell culture supernatant was loaded onto HisTrap FF 5 mL column (GE Healthcare Life Science, Cat #17-5255-01). The column was washed twice, once with binding buffer (10 mM Tris-HCl, 500 mM NaCl, pH 7.4) and then with binding buffer + 12.5 mM imidazole to remove unspecifically bound material. Finally, bound RBD protein was eluted off the column with elution buffer (10 mM Tris-HCl, 500 mM NaCl, 500 mM imidazole, pH 7.4). Column loading, washes, and elution were all performed at a 3 mL/min flow rate. After elution, the collected fraction was buffer exchanged into 1x PBS.

RBD validation by bottom-up proteomics. Fifty µg of RBD was acetone/TCA precipitated with 8 volumes of cold acetone and one volume of trichloroacetic acid overnight at -20° C. After washing the pellet with ice-cold acetone, the resulting protein pellet was resuspended into 50 µL of 8 M urea in 400 mM ammonium bicarbonate, pH 7.8, reduced with 4 mM dithiothreitol at 50° C. for 30 min., and cysteines were alkylated with 18 mM iodoacetamide in the dark for 30 min. The solution was then diluted to <2 M urea (final concentration), and trypsin (Promega) was added at a final trypsin/protein ratio of 1:50 prior to overnight incubation at 37° C. with shaking. The resulting peptides were desalted using solid-phase extraction on a Pierce C18 Spin column and eluted in 80 µL of 80% acetonitrile (ACN) in 0.1% formic acid (FA). After lyophilization, peptides were reconstituted with 5% ACN in 0.1% FA.

Peptides were analyzed by LC-MS/MS using a Dionex UltiMate 3000 Rapid Separation nanoLC and a Q Exactive™ HF Hybrid Quadrupole-Orbitrap™ Mass Spectrometer (Thermo Fisher Scientific Inc, San Jose, CA). Approximately 1 µg of peptide sample was loaded onto the trap column, which was 150 µm × 3 cm in-house packed with 3 µm ReproSil-Pur® C18 beads (Maisch, GmbH, Germany). The analytical column was a 75 µm × 10.5 cm PicoChip column packed with 3 µm ReproSil-Pur® C18 beads (New Objective, Inc. Woburn, MA). The flow rate was kept at 300 nL/min. Solvent A was 0.1% FA in water, and Solvent B was 0.1% FA in ACN. The peptides were separated on a 120 min. analytical gradient from 5% ACN/0.1% FA to 40% ACN/0.1% FA. The mass spectrometer was operated in a data-dependent mode. The source voltage was 2.40 kV, and the capillary temperature was 320° C. MS¹ scans were acquired from 300-2000 m/z at 60,000 resolving power and automatic gain control (AGC) set to 3×10⁶ charges. The top 20 most abundant precursor ions in each MS¹ scan were selected for fragmentation. Precursors were selected with an isolation width of 2 m/z and fragmented by Higher-energy collisional dissociation (HCD) at 30% normalized collision energy in the HCD cell. Previously selected ions were dynamically excluded from re-selection for 20 seconds. The MS² minimum AGC was set to 1×10³.

Proteins were identified from the tandem mass spectra extracted by Xcalibur version 4.0. MS² spectra were searched against the Spike protein RBD and SwissProt Homo sapiens database using Mascot search engine (Matrix Science, London, UK; version 2.7.0). All searches included carbamidomethyl cysteine as a fixed modification, oxidized methionine, deamidated asparagine/glutamine, and acetylated N-terminal as variable modifications. Three missed tryptic cleavages were allowed. The MS¹ precursor mass tolerance was set to 10 ppm, and the MS² tolerance was set to 0.05 Da. The search result was visualized by Scaffold v 5.0 (Proteome Software, INC., Portland, OR). A 1% false discovery rate cutoff was applied at the peptide level. Only proteins with a minimum of two unique peptides above the cutoff were considered identifications.

Fabrication of RBD-loaded magnetic beads. Recombinantly-expressed RBD was covalently loaded onto Dynabeads® MyOne™ Carboxylic Acid beads (Thermo 65011, Thermo 65012) according to the manufacturer-provided protocol for “Two-Step Coating Procedure using NHS”. In brief, for a single preparation, 1.5 mL of bead suspension was dispensed and washed twice with 1.5 mL of 25 mM MES, pH 6.0 for 10 min. at room temperature. Following washes, bead chemistry was activated by suspending the beads into 1.5 mL of freshly-prepared 50 mg/mL N-Hydroxysuccinimide (NHS), mixing in 1.5 mL of freshly-prepared 50 mg/mL 1-Ethyl-3-(3dimethylaminopropyl) carbodiimide (EDC), and incubating for 30 min. at room temperature. Following activation, the beads were again washed twice with 1.5 mL of 25 mM MES, pH 6.0 for 10 min. at room temperature. Activated beads were suspended into 1 mL 25 mM MES, pH 6.0 to which 1.5 mg of RBD protein was added. The sample was mixed and incubated for 30 min. at room temperature. Next, the beads were pulled down, the supernatant was discarded, and loading was quenched by incubating the beads in 1 mL of 50 mM Tris, pH 6.8, for 15 min. at room temperature. After quenching, beads were washed four times in 1 mL 1x PBS + 0.1% human serum albumin (HSA), and finally suspended into 3 mL 1x PBS + 0.1% HSA for storage at 4° C.

Enrichment of RBD-reactive antibodies from patient samples. Antibody pull-downs were assembled by combining 100 µL of patient plasma/serum with 35 µL of RBD-loaded bead suspension and diluting to a volume of 1 mL with 1x TBS. Assembled pull-downs were incubated overnight at 4° C. with end-over-end mixing. After this, incubation beads were pulled down, supernatant was removed, and beads were resuspended into 1 mL wash buffer (1x TBS + 0.1% TWEEN + 1% NP-40 + 1% NP-40 substitute). Suspensions were transferred onto a KingFisher Flex for additional 4 washes in 1 mL wash buffer, 2 washes in 1 mL 1x TBS, and a 30 min. incubation in 100 µL 100 mM glycine, pH 11.5 + 0.1% sodium deoxycholate at 37° C. to elute antibodies associated with bead-bound RBD. For workflow 2, Ig-pull-downs included an on-bead IdeS digestion of RBD-binding antibodies. This involved an additional incubation step between the first and second 1x TBS washes, during which beads were incubated in 100 µL of 50 mM sodium phosphate + 150 mM sodium chloride (pH 6.6) in the presence of 200 U IdeS enzyme (Promega V751A) for 3.5 h at 37° C.

Preparation of enriched antibodies for individual ion mass spectrometry. Following elution, 100 ng of mAb CR3022 standard antibody was added to each elution fraction to serve as an internal standard across all samples. For samples including an IdeS digest step, the mAb CR3022 standard underwent an in-solution IdeS digestion according to manufacturer protocols prior to being added to pull-down elution fractions. After supplementation with standard antibody, each fraction was combined with 160 µL 8 M urea and 25 µL 1 M TCEP, mixed, and set to incubate for 1 h at room temperature to facilitate the complete denaturation of the Ig-RBD and permit the complete reduction of all inter- and intrachain disulfide bonds. Following incubation, reduced antibody fragments were cleaned via methanol-chloroform-water precipitation as has been previously described^{35,36}.

Visualization of RBD-binding antibodies via western blot. Fractions of interest were combined with Bolt™ loading buffer (final concentration 1x, Thermo Fisher B0007) and dithiothreitol (DTT, final concentration 100 mM) and incubated at 100° C. for 10 min. Boiled samples were loaded onto a 4-12% Bolt™ Bis-Tris Plus polyacrylamide gel, which was run in a Mini Gel Tank (Thermo Fisher A25977) using MES running buffer for 1 h at 120 V. Next, the gel was trimmed, and proteins were transferred to a nitrocellulose membrane using iBlot™ 2 nitrocellulose transfer stacks (Thermo Fisher IB23001) on an iBlot™ 2 Dry Blotting System following manufacturer instructions. The transfer method used was the templated method P3 (20 V for 7 min.). After transfer, the membrane was blocked using 1x TBS + 0.05% TWEEN + 5% milk for 1 h at room temperature with mixing. After 1 h, Goat anti-Human IgG (H+L) HRP conjugate (Thermo Fisher 31410) was added directly to the milk at a 1:20000 dilution and the membrane was moved to 4° C. to incubate overnight. The next day, the milk + antibody mixture was discarded, and the membrane was washed 3x in 1x TBS + 0.05% TWEEN for 5 min. at room temperature with mixing. Finally, the blot was imaged on an iBright CL1000 imager (Thermo Fisher) using 800 µL of Immobilon Classico Western HRP substrate (Millipore Sigma WBLUC0500).

Western blot identification of enriched antibody isotypes. An enrichment was performed as described in the section “Enrichment of RBD-reactive antibodies from patient samples” above to screen for the presence of individual human antibody isotypes (IgG1, IgG2, IgG3, IgG4, IgA, IgD, IgE, and IgM). In this case, after elution, the fraction was divided into 8 equal parts. Positive controls were assembled for each isotype being examined, with each control consisting of 250 ng of a commercially-obtained purified antibody of that isotype (IgG1: Sigma AG502; IgG2: Sigma I5404; IgG4: Sigma I4640; IgA: Sigma I4036; IgD: Sigma 401164; IgE: Sigma 401152; IgM: Sigma I8260). Negative controls for each isotype were assembled by combining 300 ng of all commercially obtained purified antibody isotypes omitting the specific isotype that the control was. Eluted material, positive, and negative controls were run in polyacrylamide gels, and proteins were transferred to nitrocellulose and blocked as described above. After blocking, each membrane was introduced to a primary antibody that specifically recognized the isotype of the antibodies being examined on that membrane (IgG1: AbCam ab 108969; IgG2: AbCam ab 134050; IgG3: AbCam ab109761; IgG4: AbCam ab109493; IgA: AbCam ab124716; IgD: AbCam ab124795; IgE: AbCam ab195580; IgM: AbCam ab134159) mixed with 1x TBS + 0.05% TWEEN + 5% milk. Primary antibodies were present at a dilution of 1:10000. Membranes were incubated in primary antibodies overnight at 4° C. with mixing. The next day, primary antibody + milk was discarded, and all membranes were washed 3x in 1x TBS + 0.05% TWEEN for 5 min. at room temperature with mixing. After washing, all membranes were submerged into 1x TBS + 0.05% TWEEN + 5% milk to which secondary antibody (Gt-anti-Rb-HRP (Sigma AP307P) had been added at a dilution of 1:5000. Membranes incubated in secondary antibody for 1 h at room temperature with mixing, after which they were washed 3x in 1x TBS + 0.05% TWEEN for 5 min. at room temperature with mixing and imaged as described above.

Anti-RBD antibody quantification. Titers of antibodies from patient plasma responsive to SARS-CoV-2 RBD were measured using Lumit™ Dx SARS-CoV-2 Immunoassay (Promega VB1080). Plasma from each patient was diluted 1:10 in 1x TBS and incubated at 56° C. for 1 hr to inactivate any potential pathogens remaining. Following heat inactivation, samples were used as input and analyzed in triplicate using the assay following manufacturer protocols.

Individual ion mass spectrometry. After precipitation, reduced antibody samples were redissolved in 200 µL of a 40% ACN and 0.5% acetic acid. A total of 70 µL of the sample was sprayed through an Ion Max Source (Thermo Fisher Scientific) fitted with a HESI II probe at a flow rate of 1 µL/min delivered by a PAL3 robot (CTC Analytics) and analyzed by a Q Exactive Plus mass spectrometer (Thermo Fisher Scientific)^{37,38}. Instrumental conditions included eFT off, pressured setting of 0.5, 1 kV orbitrap central electrode voltage, a spray voltage of 3.0 to 4 kV, sheath gas of 2 L/min., an in-source collision-induced-dissociation value of ~15 V, and a source temperature of 320° C. Injection times ranged from 1-400 ms and were optimized on a per-sample basis to collect hundreds to thousands of individual ions (depending on spectral complexity) per acquisition event to perform I²MS analysis. Data were acquired from 650-2,500 m/z range for 70 min. using 140,000 of resolving power (at 200 m/z).

Previously, I²MS analysis has been demonstrated using an Orbitrap mass analyzer^{12,39,40}. Briefly, I²MS is a novel approach capable of discerning the mass profile of highly complex mixtures not amenable to venerable approaches used in protein mass spectrometry. Rather than measuring in the mass-to-charge (m/z) domain, I²MS accurately determines the charge on each individual ion collected through a process called STORI plot analysis. STORI plot analysis, the determination of the slope of individual ion signal accumulation, was completed on each ion as a function of its specific frequency¹². To simplify this process and reduce file sizes, only time-domain signals at specific frequencies where individual ions occurred, called STORI files, were recorded on an acquisition-to-acquisition basis. Each acquired STORI file was then processed to accurately determine the m/z and charge for every individual ion signal detected.

In order to evaluate the lower limits of the GIDI-UP platform, we analyzed NIST antibody standard in a serial dilution series. All samples were infused at 1 µL/min. for 50 min. such that the same number of transients would be acquired for each experiment. Additionally, the injection time was scaled inversely to the sample concentration in order to maintain a constant per-cycle injection of 12.5 pg. For example, the 500 nM run used an injection time of 10 ms, and the 100 nM run used an injection time of 50 ms.

Proteoform Quantification for Light Chain, Fd, and Heavy Chain. I²MS STORI files were processed to create mass spectra as .mzml files. Briefly, STORI files containing single ion peak information and transient sections are processed using a Short-time Fourier transform (STFT) to assign slopes to single ions⁴¹. After slope assignment, charges were assigned to individual ion using an iterative voting algorithm, and spectra were generated using a Normal kernel density estimate (KDE) and exported as either profile or centroided .mzml files.

Calculation of Ig-MS Ion Titer, Degree of Clonality and Spectral Correlation Coefficients. Ig-MS Ion Titers were calculated using a custom script, in which .mzml I²MS files were centroided and spectra divided into regions; the Light Chain (LC) region, the regions of the Standard peaks, and either the Fd fragment or Heavy Chain (HC) regions for samples that were reduced with and without IdeS digestion, respectively. An average noise was calculated over these regions, and the sum was subtracted from the intensity for the region’s total⁴². The titers were obtained by determining the ratio between the sum of the standard peak regions and the regions for the LC and Fd/HC regions, divided by the value of the spike-in standard, as shown in Equation 1.

$$\begin{matrix}T i t e r = \\ \frac{\begin{matrix}{\sum }_{L C m i n}^{L C m a x} \left(i n t e n s i t y - n o i s e\right) + \\ {\sum }_{F d / H C m i n}^{F d / H C m a x} \left(i n t e n s i t y - n o i s e\right)\end{matrix}}{{\sum }_{n} \left[{\sum }_{s t p e a k n m i n}^{s t d p e a k n m a x} \left(i n t e n s i t y - n o i s e\right)\right]} / s t d . m a s s \left(μ g\right)\end{matrix}$$

The degree of clonality (DoC) was calculated by centroiding profile spectra for a given mass window corresponding to the LC region. Firstly, the highest centroid peak is determined and using an averaging distribution, a window is created around the peak⁴³. To account for simple adducts (+Na and —H₂O) the window was extended +39 and - 18 Da. The intensities in this range are summed and divided by the total sum of all intensities in the LC region and inverted yield the DoC, as in Equation 2. This metric takes a value of one to infinity, with the higher the number, the more complex the spectrum.

$$D O C = \frac{{\sum }_{L C m i n}^{L C m a x} i n t e n s i t y}{{\sum }_{p e a k w i n d o w m i n}^{p e a k w i n d o w m a x} i n t e n s i t y}$$

Spectral correlation coefficients were calculated for two spectra by taking centroided spectra and creating a padded array of equal length for each. Each peak in each centroided spectra was fitted to a Normal KDE and summed into the padded arrays, yielding two gaussian fitted spectra of equal length with indexes corresponding to the same mass. Calculating the Cosine Similarity using Equation 3, yielded the spectral correlation coefficients of the two spectra.

$$s i m i l a r i t y = \frac{A ⋅ B}{\left‖A\right‖ \left‖B\right‖} = \frac{{\sum }_{i = 1}^{n} {A}_{i} {B}_{i}}{\sqrt{{\sum }_{i = 1}^{n} {A}_{i}^{2} \sqrt{{\sum }_{i = 1}^{n} {B}_{i}^{2}}}}$$

IgG Antibody glycan analysis. Reduced IgG purified from plasma were separated on an SDS-PAGE gel and the band correspondent to the heavy chain was cut and subjected to in-gel digestion as follows: gel bands were washed in 100 mM Ammonium Bicarbonate (AmBic)/Acetonitrile (ACN) and reduced with 10 mM dithiothreitol at room temperature for 45 min. Cysteines were alkylated with 50 mM iodoacetamide in the dark for 45 min. at room temperature. Finally, gel bands were washed in 100 mM AmBic/ACN prior to adding 600 ng Lys-C for overnight incubation at room temperature. Following digest, supernatants containing peptides were transferred into new tubes. Gel pieces were washed at room temperature for 10 min. with gentle shaking, in 50% ACN/5% FA, and supernatants were combined with peptide solutions. This wash was repeated each by 80% ACN/5% FA, and 100% ACN, and all supernatants was saved. Pooled supernatants were then subject to speedvac drying. After lyophilization, peptides were reconstituted with 5% ACN/0.1% FA in water.

Peptides were analyzed by LC-MS/MS using a Dionex UltiMate 3000 Rapid Separation nanoLC and a Q Exactive™ HF Hybrid Quadrupole-Orbitrap™ Mass Spectrometer (Thermo Fisher Scientific Inc). The peptide samples were loaded onto the trap column, which was 150 µm × 3 cm in-house packed with 3 µm C18 beads. The analytical column was a 75 µm × 10.5 cm PicoChip column packed with 3 µm C18 beads (New Objective). The flow rate was kept at 300 nL/min. Solvent A was 0.1% FA in water and Solvent B was 0.1% FA in ACN. The peptide was separated on a 60 min. analytical gradient from 5% to 50% of Solvent B. The mass spectrometer was operated in the Full MS scan. The source voltage was 2.30 ~ 2.50 kV and the capillary temperature 320° C. Full MS scans were acquired from 400-2000 m/z at 60,000 resolving power and automatic gain control (AGC) set to 3x10⁶.

MS data were processed using Skyline (Version 20.2). The integration and correction for the chromatographic peaks of 18 glycans were performed manually. Three of the most intense precursor ions of each glycan were selected, summed, and exported as the quantitative value of the corresponding glycan. Total ion intensities were used to generate the plots.

Statistical analysis. ANOVA statistics were calculated using SAS (SAS Institute, Cary, NC). Person’s correlations were calculated using the function “cor.test” and method “pearson” on RStudio v3.1.1073. Violin and dot plots were generated with GraphPad Prism 9.0.2 and that same software was used for calculating the statistical significance by one-way ANOVA with Tukey’s multiple comparison test (* p<0.05; ** p<0.01).

### Example 5

The presently disclosed methods may also be used to for monitoring or detecting the presence of an autoimmune disorder. In addition, the readouts generated by the methods disclosed herein may also provide for a qualitative biomarker or unique peak or feature that may characterize the presence or severity of the autoimmune disorder.

FIG. 24 shows Ig-MS spectra from Myasthenia Gravis (MYG) patients versus a healthy donor with MuSK as the proposed antigen. The presence of peak 2 in the Ig-MS spectrum of patient MYG69 with MuSK MG provides a qualitative biomarker of the disease.

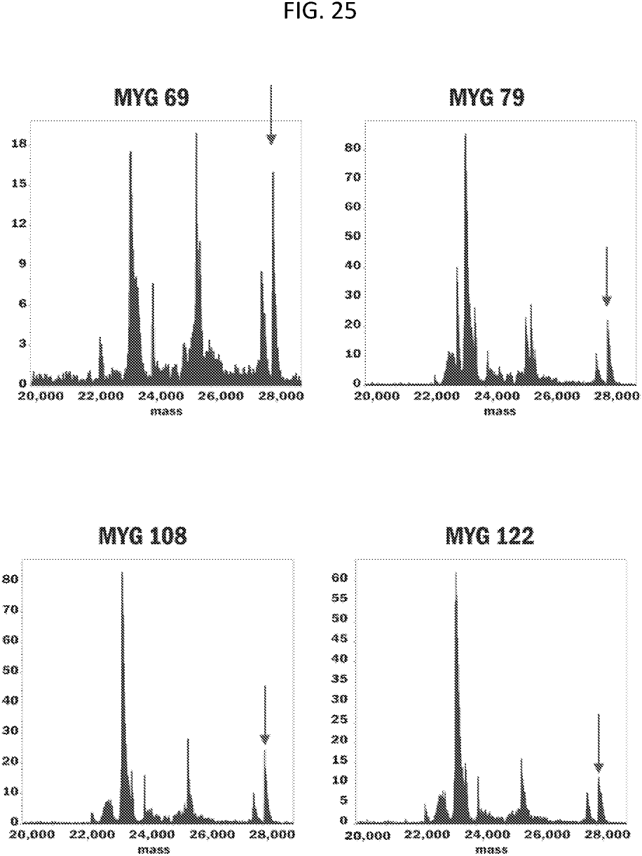

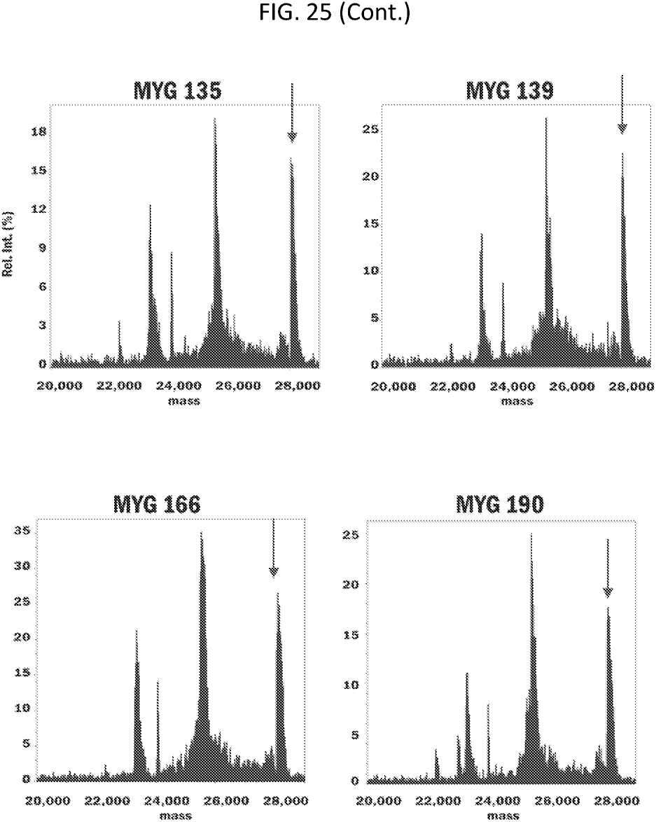

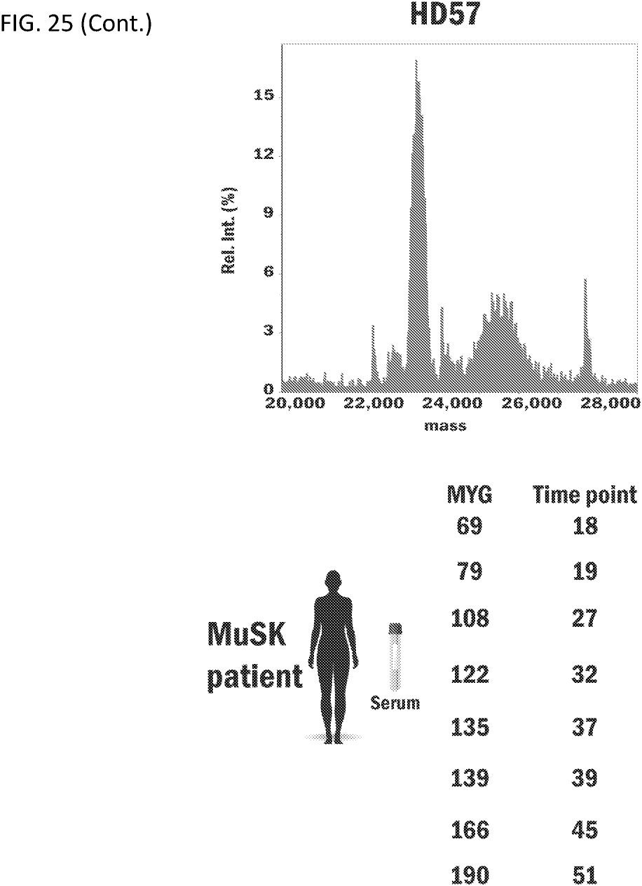

FIG. 25 shows Ig-MS spectra from MYG patients and a healthy donor at various time points. The Ig-MS spectra obtained from MYG patients etains the extra peak, indicated by an arrow, over several months.

### REFERENCES

1 Zhou, P. et al. A pneumonia outbreak associated with a new coronavirus of probable bat origin. Nature 579, 270-273, doi:10.1038/s41586-020-2012-7 (2020).

2 Zhou, Y. et al. Comorbidities and the risk of severe or fatal outcomes associated with coronavirus disease 2019: A systematic review and meta-analysis. Int J Infect Dis 99, 47-56, doi:10.1016/j.ijid.2020.07.029 (2020).

3 Ahmad, F. B., Cisewski, J. A., Minino, A. & Anderson, R. N. Provisional Mortality Data - United States, 2020. MMWR Morb Mortal Wkly Rep 70, 519-522, doi:10.15585/mmwr.mm7014el (2021).

4 Wang, P. et al. Antibody resistance of SARS-CoV-2 variants B.1.351 and B.1.1.7. Nature, doi:10.1038/s41586-021-03398-2 (2021).

5 Wu, J. et al. SARS-CoV-2 infection induces sustained humoral immune responses in convalescent patients following symptomatic COVID-19. Nat Commun 12, 1813, doi:10.1038/s41467-021-22034-1 (2021).

6 Kreer, C. et al. Longitudinal Isolation of Potent Near-Germline SARS-CoV-2-Neutralizing Antibodies from COVID-19 Patients. Cell 182, 1663-1673, doi:10.1016/j.cell.2020.08.046 (2020).

7 Rogers, T. F. et al. Isolation of potent SARS-CoV-2 neutralizing antibodies and protection from disease in a small animal model. Science 369, 956-963, doi:10.1126/science.abc7520 (2020).

8 Wheatley, A. K. et al. Evolution of immune responses to SARS-CoV-2 in mild-moderate COVID-19. Nat Commun 12, 1162, doi:10.1038/s41467-021-21444-5 (2021).

9 Lee, J. et al. Molecular-level analysis of the serum antibody repertoire in young adults before and after seasonal influenza vaccination. Nat Med 22, 1456-1464, doi:10.1038/nm.4224 (2016).

10 Smith, L. M., Kelleher, N. L. & Consortium for Top Down, P. Proteoform: a single term describing protein complexity. Nat Methods 10, 186-187, doi:10.1038/nmeth.2369 (2013).

11 He, L. et al. Analysis of Monoclonal Antibodies in Human Serum as a Model for Clinical Monoclonal Gammopathy by Use of 21 Tesla FT-ICR Top-Down and Middle-Down MS/MS. J Am Soc Mass Spectrom 28, 827-838, doi:10.1007/s13361-017-1602-6 (2017).

12 Kafader, J. O. et al. Multiplexed mass spectrometry of individual ions improves measurement of proteoforms and their complexes. NatMethods 17, 391-394, doi:10.1038/s41592-020-0764-5 (2020).

13 Amanat, F. et al. A serological assay to detect SARS-CoV-2 seroconversion in humans. Nat Med 26, 1033-1036, doi:10.1038/s41591-020-0913-5 (2020).

14 Barnidge, D. R. et al. Phenotyping Polyclonal Kappa and Lambda Light Chain Molecular Mass Distributions in Patient Serum Using Mass Spectrometry. Journal of Proteome Research 13, 5198-5205, doi:10.1021/pr5005967 (2014).

15 Klein, S. L. et al. Sex, age, and hospitalization drive antibody responses in a COVID-19 convalescent plasma donor population. J Clin Invest 130, 6141-6150, doi:10.1172/JCI142004 (2020).

16 Lynch, K. L. et al. Magnitude and Kinetics of Anti-Severe Acute Respiratory Syndrome Coronavirus 2 Antibody Responses and Their Relationship to Disease Severity. Clinical Infectious Diseases 72, 301-308, doi:10.1093/cid/ciaa979 (2020).

17 Amanat, F. et al. A serological assay to detect SARS-CoV-2 seroconversion in humans. Nature Medicine 26, 1033-1036, doi:10.1038/s41591-020-0913-5 (2020).

18 Gunn, B. M. et al. Enhanced binding of antibodies generated during chronic HIV infection to mucus component MUC16. Mucosal Immunology 9, 1549-1558, doi:10.1038/mi.2016.8 (2016).

19 Larsen, M. D. et al. Afucosylated IgG characterizes enveloped viral responses and correlates with COVID-19 severity. Science 371, eabc8378, doi:10.1126/science.abc8378 (2021).

20 Ferrara, C. et al. Unique carbohydrate-carbohydrate interactions are required for high affinity binding between FcyRIII and antibodies lacking core fucose. Proceedings of the National Academy of Sciences, doi:10.1073/pnas.1108455108 (2011).

21 Lau, E. H. Y. et al. Neutralizing antibody titres in SARS-CoV-2 infections. Nat Commun 12, 63, doi:10.1038/s41467-020-20247-4 (2021).

22 Klassen, S. et al. Convalescent Plasma Therapy for COVID-19: A Graphical Mosaic of the Worldwide Evidence. SSRN, doi:https://dx.doi.org/10.2139/ssrn.3806768 (2021).

23 Galson, J. D. et al. Deep Sequencing of B Cell Receptor Repertoires From COVID-19 Patients Reveals Strong Convergent Immune Signatures. Frontiers in Immunology 11, doi:10.3389/fimmu.2020.605170 (2020).

24 Robbiani, D. F. et al. Convergent antibody responses to SARS-CoV-2 in convalescent individuals. Nature 584, 437-442, doi:10.1038/s41586-020-2456-9 (2020).

25 Cheung, W. C. et al. A proteomics approach for the identification and cloning of monoclonal antibodies from serum. Nat Biotechnol 30, 447-452, doi:10.1038/nbt.2167 (2012).

26 Wine, Y. et al. Molecular deconvolution of the monoclonal antibodies that comprise the polyclonal serum response. Proc Natl Acad Sci USA 110, 2993-2998, doi:10.1073/pnas. 1213737110 (2013).

27 Tran, N. H. et al. Complete De Novo Assembly of Monoclonal Antibody Sequences. Sci Rep 6, 31730, doi:10.1038/srep31730 (2016).

28 Guthals, A. et al. De Novo MS/MS Sequencing of Native Human Antibodies. J Proteome Res 16, 45-54, doi:10.1021/acs.jproteome.6b00608 (2017).

29 Georgiou, G. et al. The promise and challenge of high-throughput sequencing of the antibody repertoire. Nat Biotechnol 32, 158-168, doi:10.1038/nbt.2782 (2014).

30 Fornelli, L. et al. Accurate Sequence Analysis of a Monoclonal Antibody by Top-Down and Middle-Down Orbitrap Mass Spectrometry Applying Multiple Ion Activation Techniques. Anal Chem 90, 8421-8429, doi:10.1021/acs.analchem.8b00984 (2018).

31 Liu, L. et al. Potent neutralizing antibodies against multiple epitopes on SARS-CoV-2 spike. Nature 584, 450-456, doi:10.1038/s41586-020-2571-7 (2020).

32 Yamayoshi, S. et al. Antibody titers against SARS-CoV-2 decline, but do not disappear for several months. EClinicalMedicine 32, 100734, doi:10.1016/j.eclinm.2021.100734 (2021).

33 Lopez-Santibanez-Jacome, L., Avendano-Vazquez, S. E. & Flores-Jasso, C. F. The Pipeline Repertoire for Ig-Seq Analysis. Front Immunol 10, 899, doi:10.3389/fimmu.2019.00899 (2019).

34 Stadlbauer, D. et al. SARS-CoV-2 Seroconversion in Humans: A Detailed Protocol for a Serological Assay, Antigen Production, and Test Setup. Curr Protoc Microbiol 57, e100, doi:10.1002/cpmc.100 (2020).

35 Toby, T. K. et al. A comprehensive pipeline for translational top-down proteomics from a single blood draw. Nat Protoc 14, 119-152, doi:10.1038/s41596-018-0085-7 (2019).

36 Wessel, D. & Flugge, U. I. A method for the quantitative recovery of protein in dilute solution in the presence of detergents and lipids. Anal Biochem 138, 141-143, doi:10.1016/0003-2697(84)90782-6 (1984).

37 Skinner, O. S. et al. An informatic framework for decoding protein complexes by top-down mass spectrometry. Nat Methods 13, 237-240, doi:10.1038/nmeth.3731 (2016).

38 Park, H.-M. et al. Novel Interface for High-Throughput Analysis of Biotherapeutics by Electrospray Mass Spectrometry. Analytical chemistry 92, 2186-2193, doi:10.1021/acs.analchem.9b04826 (2020).

39 Kafader, J. O. et al. Individual Ion Mass Spectrometry Enhances the Sensitivity and Sequence Coverage of Top-Down Mass Spectrometry. Journal of Proteome Research 19, 1346-1350, doi:10.1021/acs.jproteome.9b00797 (2020).

40 McGee, J. P. et al. Isotopic Resolution of Protein Complexes up to 466 kDa Using Individual Ion Mass Spectrometry. Analytical Chemistry 93, 2723-2727, doi:10.1021/acs.analchem.0c03282 (2021).

41 Kafader, J. O. et al. STORI Plots Enable Accurate Tracking of Individual Ion Signals. J Am Soc Mass Spectrom 30, 2200-2203, doi:10.1007/s13361-019-02309-0 (2019).

42 Horn, D. M., Zubarev, R. A. & McLafferty, F. W. Automated reduction and interpretation of high resolution electrospray mass spectra of large molecules. J Am Soc Mass Spectrom 11, 320-332, doi:10.1016/s1044-0305(99)00157-9 (2000).

43 Senko, M. W., Beu, S. C. & McLafferty, F. W. Determination of monoisotopic masses and ion populations for large biomolecules from resolved isotopic distributions. Journal of the American Society for Mass Spectrometry 6, 229-233, doi:https://doi.org/10.1016/10440305(95)00017-8 (1995).

1. A method for immunoglobulin repertoire profiling, the method comprising:
reducing a mixture of immunoglobulins in a sample, the sample optionally further comprising a standard immunoglobulin, thereby obtaining a second sample comprising a mixture of light chains and heavy chains or light chains and Fd domains;
ionizing the second sample with an ionizer;
detecting a multiplicity of ions generated by the ionization of the second sample with a current detector;
determining ion masses for each of the multiplicity of ions detected with the current detector with a mass analyzer;
generating a mass-domain spectrum from the ion masses with the mass analyzer; and
determining one or more metrics capturing the heterogeneity or relative abundance of individual immunoglobulins.

2. The method of claim 1, wherein an Ion Titer (IT), a degree of clonality (DoC), a spectral correlation coefficient, or any combination thereof is determined.

3. The method of claim 2, wherein the Ion Titer (IT) is determined by a ratio between (i) a sum of mass-domain peak intensities across a light chain region and mass-domain peak intensities across a heavy chain region or Fd region and (ii) a sum of mass-domain peak intensities across a standard region divided by the value of the standard immunoglobulin.

4. The method of claim 2, wherein the degree of clonality (DoC) is determined by a ratio between a sum of mass-domain peak intensities across a light chain region and a sum of mass-domain peak intensities across a peak window.

5. The method of any one of claims 1-4, further comprising fractionating the sample prior to reduction.

6. The method of claim 5, wherein the sample is fractionated by immunoprecipitation.

7. The method of any one of claims 1-6, wherein the mixture of different immunoglobulins in the sample are chemically modified prior to ionization.

8. The method of claim 7, wherein the chemical modification is digestion with a protease.

9. The method of any one of claims 1-8, wherein the method further comprises collecting a sample from a subject or a candidate donor.

10. A method of identifying a donor comprising the method according to any one of claims 1-8, wherein the sample has been collected from a candidate donor and wherein the donor is identified from the one or more metrics capturing the heterogeneity or relative abundance of individual immunoglobulins.

11. A method for obtaining a target immunoglobulin comprising the method according to claim 10 and further comprising obtaining a target immunoglobulin from the donor.

12. The method of claim 11, further comprising isolating and/or purifying the target immunoglobulin.

13. A method of preparing a proteome product comprising the method according to any one of claims 11-12 and further comprising contacting the target immunoglobulin obtained to the donor with a pharmaceutically acceptable excipient, diluent, carrier, stabilizer, anticoagulant, or any combination thereof.

14. A method of treating a subject in need of a proteome product, the method comprising administering an effective amount of the proteome product prepared by the method according to claim 13 to the subject.

15. The method of claim 14, wherein the subject in need of an immunoglobulin therapy.

16. The method of any one of claims 14-15, wherein the subject has an infection.

17. A proteome product prepared by the method according to claim 13.

18. A method for identifying a target proteoform or a complex thereof, the method comprising:
ionizing a sample with an ionizer, wherein the sample comprises a mixture of different proteoforms or complexes thereof;
detecting a multiplicity of ions generated by the ionization of the sample with a current detector;
determining ion masses for each of the multiplicity of ions detected with the current detector with a mass analyzer;
generating a mass-domain spectrum from the ion masses with the mass analyzer;
obtaining a spectral signature for a target ion mass of the target proteoform or complex thereof with a gene analyzer; and
identifying the presence or absence of the spectral signature for the target ion mass in the mass-domain spectrum.

19. The method of claim 18, wherein the target proteoform is a full-length target proteoform.

20. The method of any one of claims 18-19, wherein obtaining the spectral signature comprises determining a mass of the target proteoform or the complex thereof from a nucleic acid sequence corresponding to the target proteoform or the complex thereof.

21. The method of claim 20, wherein the nucleic acid sequences are obtained from the sample.

22. The method of claim 20, wherein the nucleic acid sequences are obtained from a database.

23. The method of any one of claims 18-22,
wherein detecting the multiplicity of ions generated by the ionization of the sample comprises detecting an ion frequency signal, an ion intensity signal, an ion mass-to-charge ratio (m/z) signal, and an induced current as a function of time and
wherein determining the ion masses for each fo the multiplicity of ions detected comprises determining the ion masses for each of the multiplicity of ions detected with the current detector from the ion frequency signal, the ion intensity signal, the ion mass-to-charge ratio (m/z) signals, and the induced current as a function of time.

24. The method of any one of claim 18-23, wherein the target proteoform or a complex thereof is an immunoglobulin, a heavy chain thereof, a light chain thereof, or any combination thereof.

25. The method of any one of claims 18-24, wherein the method further comprises collecting a sample from a subject or a candidate donor.

26. The method of any one of any one of claims 18-24, wherein the method further comprises fractionating the sample prior to ionization.

27. The method of claim 26, wherein the sample is fractionated by centrifugation, precipitation, size exclusion, binding affinity, or any combination thereof.

28. The method of any one of any one of claims 18-27, wherein the mixture of different full-length proteoforms or complexes thereof in the sample are chemically modified prior to ionization.

29. The method of claim 28, wherein the chemical modification is disulfide reduction, deglycosylation, digestion with a protease, or any combination thereof.

30. The method of any of the preceding claims, wherein the method further comprises isolating one or more cells from the sample comprising the target proteoform or a complex thereof and sequencing a nucleic acid from the isolated cells, wherein the sequenced nucleic acid corresponds to the target proteoform or a complex thereof.

31. The method of claim 30, wherein the one or more cells are PBMCs.

32. A method of identifying a donor comprising the method according to any one of claims 18-31, wherein the sample has been collected from a candidate donor and wherein the donor is identified by the presence of the spectral signature for the target ion mass in the mass-domain spectrum.

33. A method for obtaining a target proteoform comprising the method according to claim 32 and further comprising obtaining the target proteoform from the donor.

34. The method of claim 33, further comprising isolating and/or purifying the target immunoglobulin.

35. A method of preparing a proteome product comprising the method according to any one of claims 33-34 and further comprising contacting the target proteome or the complex thereof obtained to the donor with a pharmaceutically acceptable excipient, diluent, carrier, stabilizer, anticoagulant, or any combination thereof.

36. A method of treating a subject in need of a proteome product, the method comprising administering an effective amount of the proteome product prepared by the method according to claim 35 to the subject.

37. The method of claim 36, wherein the subject in need of an immunoglobulin therapy.

38. The method of any one of claims 15-36, wherein the subject has an infection.

39. A proteome product prepared by the method according to claim 35.


SEQUENCE LISTING (4 sequences)

SEQ ID NO: 1 - DNA, 91 bp, Artificial Sequence
  Synthetic - Exemplary immunoglobulin gene sequence shown in FIG. 1
atatatgaaagtatatataatttaatcttttttctttttagcgcgcacccgcgcacacagaaacactctctccccctcgagagagggggag

SEQ ID NO: 2 - protein, 11 aa, Artificial Sequence
  Synthetic - Exemplary peptide fragment shown in FIG. 3
ARDLVVYGMDV

SEQ ID NO: 3 - protein, 11 aa, Artificial Sequence
  Synthetic - Position weight matrix shown in FIG. 6
  MISC_FEATURE (4)..(4): X is L, A, R, P, or T
  MISC_FEATURE (5)..(5): X is V, I, M, or Q
  MISC_FEATURE (6)..(6): X is V, S, M, N, or R
  MISC_FEATURE (7)..(7): X is Y, G, N, or R
ARDXXXXGMDV

SEQ ID NO: 4 - protein, 243 aa, Artificial Sequence
  Synthetic - Spike protein receptor binding domain shown in FIG. 8A
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH